(19) United States
(12) Patent Application Publication
(10) Pub. No.: US 2020/0176101 A1
(43) Pub. Date: Jun. 4, 2020

(54) METHODS AND MEDICAL USES RELATING TO THE TREATMENT OF HYPOGLYCAEMIA

(71) Applicants: Zealand Pharma A/S, Søborg (DK); Hvidovre Hospital, Hvidovre (DK); Technical University of Denmark, Kgs. Lyngby (DK)

(72) Inventors: Sabrina AANÆS WENDT, Søborg (DK); Ajenthen RANJAN, Søborg (DK); Henrik MADSEN, Søborg (DK); Kirsten NØRGAARD, Søborg (DK); John BAGTERP JØRGENSEN, Søborg (DK); Signe SCHMIDT, Søborg (DK)

(21) Appl. No.: 16/630,034

(22) PCT Filed: Jul. 4, 2018
(86) PCT No.: PCT/EP2018/068085

§ 371 Date: Jan. 10, 2020

(30) Foreign Application Priority Data
Jul. 5, 2017 (GB) 1710822.6

(51) Int. Cl. G16H 20/17; G16H 70/20

(52) U.S. Cl. CPC G16H 20/17; G16H 70/20

(57) ABSTRACT

Methods and medical uses for determining a dose for a glucagon bolus for administration to patients with diabetes for treating mild or moderate hypoglycaemia, while reducing the risk of, or avoiding, rebound hyperglycaemia are described. This work is based on simulations using pharmacokinetic (PK) and pharmacodynamic (PD) models for glucose, insulin and glucagon to develop an optimum glucagon dosing regimen for treatment of mild or moderate hypoglycaemia depending on ambient insulin levels, while reducing the risk of, or avoiding, rebound hyperglycaemia, for example as may occur when an overly large dose of glucagon is administered to a patient having a hypoglycaemic episode.

### FIELD OF THE INVENTION

The present invention relates methods and medical uses for determining a dose for a glucagon bolus for administration to patients with diabetes for treating mild or moderate hypoglycaemia, while reducing the risk of, or avoiding, rebound hyperglycaemia.

### BACKGROUND OF THE INVENTION

Human preproglucagon is a 158 amino acid precursor polypeptide that is differentially processed in the tissues to form a number of structurally related proglucagon-derived peptides, including glucagon (Glu or GCG), glucagon-like peptide-1 (GLP-1), glucagon-like peptide-2 (GLP-2), and oxyntomodulin (OXM). These molecules are involved in a wide variety of physiological functions, including glucose homeostasis, insulin secretion, gastric emptying and intestinal growth, as well as regulation of food intake.

Native glucagon is a 29-amino acid peptide that corresponds to amino acids 53 to 81 of pre-proglucagon. Glucagon helps maintain the level of glucose in the blood by binding to glucagon receptors on hepatocytes, causing the liver to release glucose—stored in the form of glycogen—through glycogenolysis. As these stores become depleted, glucagon also stimulates the liver to synthesize additional glucose by gluconeogenesis. This glucose is released into the bloodstream, preventing the development of hypoglycaemia. WO 2014/016300 (Zealand Pharma A/S) describes stable glucagon analogues and their use for the treatment of hypoglycaemia.

Owing to the relatively low physical and chemical stability of native glucagon per se, glucagon products that are currently available commercially, and which are intended primarily for use in “rescue” situations for alleviating acute and severe hypoglycaemia in a diabetic subject who has received an excessively high dose of insulin or through exercise or other factors, are provided in the form of freeze-dried, solid preparations intended for reconstitution in an appropriate liquid medium immediately before use. Hypoglycemic subjects may, inter alia, exhibit dizziness and/or confusion, and in some cases may become unconscious or semi-conscious, rendering them unable to carry out or complete the required initial liquid reconstitution and subsequent injection of the glucagon formulation in question.

In particular, intensive insulin therapy increases the risk of hypoglycaemia in patients with type 1 diabetes. The fear of hypoglycaemia impedes many in seeking optimum glycaemic control and affects their quality of life negatively. However, insulin pumps and continuous glucose measurements are treatment tools reducing occurrence of hypoglycaemia, but have not removed the fear or occurrence of hypoglycaemia completely. Different adjunct therapies and advances in insulin therapy have been tested, but none markedly improved glycaemic control, risk of hypoglycaemia and/or quality of life.

It has been suggested that low-dose glucagon as an add-on to the intensified insulin therapy may optimise glycaemic control and reduce the risk of hypoglycaemia. This dual-hormone approach has mainly been tested in settings with automatic delivery of the drugs (closed-loop therapy). However, manual delivery of insulin and glucagon (open-loop therapy) may equally improve diabetes management, which has been demonstrated in children with type 1 diabetes and gastroenteritis.

However, it remains a problem to determine the optimum dose of glucagon for treating hypoglycaemia while minimising the risk of rebound hyperglycaemia.

### SUMMARY OF THE INVENTION

Broadly, the present invention relates to methods and uses for determining an optimum dose of a glucagon bolus for treating mild or moderate hypoglycaemia in patients having diabetes, for example for use in an open-loop setting for treating type 1 diabetes. At present, patients having mild or moderate hypoglycaemic episodes are recommended to eat a snack containing glucose to ameliorate the effects of the hypoglycaemia. In part, this is a consequence of the fact that the anti-hypoglycaemic effect of glucagon is highly dependent on ambient insulin levels, and there are no commercially available devices able to measure insulin concentrations in real-time or to calculate an appropriate rescue dose of glucagon for administration to patients, that is a glucagon dose that is sufficient to correct the hypoglycaemic episode, while reducing the risk of, or avoiding, hyperglycaemia caused by too large a dose being administered to the patient. Bolus calculators in insulin pumps have addressed this issue by providing “insulin on board” (IOB) feedback to reduce the risk of insulin stacking and hypoglycaemia. Basal insulin is not included in the calculation of IOB, which is an approximation of the remaining effect of an insulin bolus, measured in units of subcutaneously (SC) administered bolus insulin.

Accordingly, the present invention is based on simulations using pharmacokinetic (PK) and pharmacodynamic (PD) models for glucose, insulin and glucagon to develop an optimum glucagon dosing regimen for treatment of mild or moderate hypoglycaemia depending on ambient insulin levels, while reducing the risk of, or avoiding, rebound hyperglycaemia, for example as may occur when an overly large dose of glucagon is administered to a patient having a hypoglycaemic episode. The work described herein used a validated glucoregulatory model to simulate how different insulin levels would affect the glucose response to different glucagon doses and the success of each glucagon dose in treating mild hypoglycaemia was evaluated. The criteria for the optimum glucagon dose to treat mild hypoglycaemia at varying insulin levels was the lowest dose that in most patients caused a plasma glucose concentration (PG) peak between 5.0 and 10.0 mmol/l and sustained PG above or equal to 3.9 mmol/l for 2 hours after the bolus. The model-based glucagon regimen of the present invention is therefore the first attempt to develop an insulin-dependent glucagon dosing regimen for treatment of insulin-induced mild hypoglycaemia in diabetic patients.

Accordingly, in a first aspect, the present invention provides an automated or computer implemented method for determining a dose for a glucagon bolus for administration to a patient with diabetes for treating mild or moderate hypoglycaemia, the method comprising:
(a) determining an ambient insulin level for the patient, wherein the ambient insulin level is directly measured by a blood sample, measured by an insulin sensor and/or approximated by active insulin on board;
(bi) using the ambient insulin level to determine the dose for the glucagon bolus to treat mild or moderate hypoglycaemia while reducing the risk of, or avoiding, rebound hyperglycaemia according to treatment criteria (1) to increase plasma glucose (PG)≥5 mmol/l, (2) to have a peak plasma glucose (PG)≤10 mmol/l, and (3) to keep plasma glucose (PG)≥3.9 mmol/l for 120 min after the glucagon bolus;
(bii) using pharmacokinetic/pharmacodynamics (PK/PD) models for simulation of a virtual patient population of diabetes patients receiving the glucagon bolus to correct insulin-induced mild or moderate hypoglycaemia, wherein the dose of the glucagon bolus is the lowest glucagon dose yielding the maximal weighted success rate in the population calculated according to said treatment criteria; and
(c) selecting the lowest dose for the glucagon bolus that provides the maximal weighted success rate according to the treatment criteria; and
(d) optionally administering the glucagon bolus to the patient to treat the hypoglycaemia.

In one aspect, the present invention provides an automated or computer implemented method for determining a dose for a glucagon bolus for administration to a patient with diabetes for treating mild or moderate hypoglycaemia, the method comprising:
(a) determining an ambient insulin level for the patient, wherein the ambient insulin level is directly measured by a blood sample, measured by an insulin sensor and/or approximated by active insulin on board;
(bi) using the ambient insulin level to determine the dose for the glucagon bolus to treat mild or moderate hypoglycaemia while reducing the risk of, or avoiding, rebound hyperglycaemia;
(bii) using pharmacokinetic/pharmacodynamics (PK/PD) models for simulation of a virtual patient population of diabetes patients receiving the glucagon bolus to correct insulin-induced mild or moderate hypoglycaemia, wherein the dose of the glucagon bolus is the lowest glucagon dose yielding the maximal weighted success rate in the population calculated according to said treatment criteria; and
(c) selecting the lowest dose for the glucagon bolus that provides the maximal weighted success rate according to the treatment criteria; and
(d) optionally administering the glucagon bolus to the patient to treat the hypoglycaemia.

In some embodiments, the methods and medical uses of the present invention include the further step of (d) administering the glucagon dose to the patient to treat the hypoglycaemia. The methods and uses are particularly suited for the treatment of hypoglycaemia in type 1 diabetes, but alternatively could also be applied to the treatment of patients having type 2 diabetes.

In a further aspect, the present invention provides a method for determining a dose for a glucagon bolus for administration to a patient with diabetes for treating mild or moderate hypoglycaemia, the method comprising:
(a) determining an ambient insulin level for the patient, wherein the ambient insulin level is directly measured by a blood sample, measured by an insulin sensor and/or approximated by active insulin on board;
(bi) using the ambient insulin level to determine the dose for the glucagon bolus to treat mild or moderate hypoglycaemia while reducing the risk of, or avoiding, rebound hyperglycaemia;
(bii) using pharmacokinetic/pharmacodynamics (PK/PD) models for simulation of a virtual patient population of diabetes patients receiving the glucagon bolus to correct insulin-induced mild or moderate hypoglycaemia, wherein the dose of the glucagon bolus is the lowest glucagon dose yielding the maximal weighted success rate in the population calculated according to said treatment criteria; and
(c) selecting the lowest dose for the glucagon bolus that provides the maximal weighted success rate according to the treatment criteria; and
(d) administering the glucagon bolus to the patient to treat the hypoglycaemia.

In some embodiments, the methods and medical uses of the present invention may include an initial step of administering insulin to the patient, optionally following the consumption of food by the patient, i.e. where the hypoglycaemia is insulin induced.

In a further aspect, the present invention provides a glucagon bolus for use in a method of treating mild or moderate hypoglycaemia in a patient with diabetes, wherein the method comprises calculating a dose for the glucagon bolus using an automated or computer implemented method which comprises:
(a) determining an ambient insulin level for the patient, wherein the ambient insulin level is directly measured by a blood sample, measured by an insulin sensor and/or approximated by active insulin on board;
(bi) using the ambient insulin level to determine the dose for the glucagon bolus to treat mild or moderate hypoglycaemia while reducing the risk of, or avoiding, rebound hyperglycaemia according to treatment criteria (1) to increase plasma glucose (PG)≥5 mmol/l, (2) to have a peak plasma glucose (PG)≤10 mmol/l, and (3) to keep plasma glucose (PG)≥3.9 mmol/l for 120 min after the glucagon bolus;
(bii) using pharmacokinetic/pharmacodynamics (PK/PD) models for simulation of a virtual patient population of diabetes patients receiving the glucagon bolus to correct insulin-induced mild or moderate hypoglycaemia, wherein the dose of the glucagon bolus is the lowest glucagon dose yielding the maximal weighted success rate in the population calculated according to said treatment criteria; and
(c) selecting the lowest dose for the glucagon bolus that provides the maximal weighted success rate according to the treatment criteria; and
(d) administering the glucagon bolus to the patient to treat the hypoglycaemia.
   In a further aspect, the present invention provides use of a glucagon bolus in the preparation of a medicament for treating mild or moderate hypoglycaemia in a patient with type 1 diabetes, wherein the method comprises calculating a dose for the glucagon bolus using a computer implemented method which comprises:
(a) determining an ambient insulin level for the patient, wherein the ambient insulin level is directly measured by a blood sample, measured by an insulin sensor and/or approximated by active insulin on board;
(bi) using the ambient insulin level to determine the dose for the glucagon bolus to treat mild or moderate hypoglycaemia while reducing the risk of, or avoiding, rebound hyperglycaemia according to treatment criteria (1) to increase plasma glucose (PG)≥5 mmol/l, (2) to have a peak plasma glucose (PG)≤10 mmol/l, and (3) to keep plasma glucose (PG)≥3.9 mmol/l for 120 min after the glucagon bolus;
(bii) using pharmacokinetic/pharmacodynamics (PK/PD) models for simulation of a virtual patient population of diabetes patients receiving the glucagon bolus to correct insulin-induced mild or moderate hypoglycaemia, wherein the dose of the glucagon bolus is the lowest glucagon dose yielding the maximal weighted success rate in the population calculated according to said treatment criteria; and
(c) selecting the lowest dose for the glucagon bolus that provides the maximal weighted success rate according to the treatment criteria.

In one aspect, the present invention provides a glucagon for use in a method of treating mild or moderate hypoglycaemia in a patient with diabetes, wherein the method comprises calculating a dose for a glucagon bolus using an automated or computer implemented method which comprises:
(a) determining an ambient insulin level for the patient, wherein the ambient insulin level is directly measured by a blood sample, measured by an insulin sensor and/or approximated by active insulin on board;
(bi) using the ambient insulin level to determine the dose for a glucagon bolus to treat mild or moderate hypoglycaemia while reducing the risk of, or avoiding, rebound hyperglycaemia;
(bii) using pharmacokinetic/pharmacodynamics (PK/PD) models for simulation of a virtual patient population of diabetes patients receiving the glucagon bolus to correct insulin-induced mild or moderate hypoglycaemia, wherein the dose of the glucagon bolus is the lowest glucagon dose yielding the maximal weighted success rate in the population calculated according to said treatment criteria; and
(c) selecting the lowest dose for the glucagon bolus that provides the maximal weighted success rate according to the treatment criteria; and
(d) administering the glucagon bolus to the patient to treat the hypoglycaemia.

In some embodiments, the method and medical uses of the present invention include the step of determining the ambient insulin level as a function of the insulin-on-board (IOB) for the patient. This may involve determining insulin-on-board (IOB) using a bolus calculator, for example a bolus calculator that uses a linear or curvilinear time profile.

Conveniently, the methods and medical uses of the present invention may be carried out using an app on a mobile device, such as a smart phone or using a device with built in processors such as an insulin pump. The methods and medical uses of the present invention can output the results of determining the optimum glucagon dose wirelessly to any convenient output device known in the art, such as a personal “smart” device (e.g. smart phone, wrist-band or smart watch), tablet or other computer, thereby instructing the patient to administer the glucagon dose for correcting the hypoglycaemic episode. Other embodiments may involve feedback to a pump capable of administering the glucagon. In either setting, the output may be a simple display of the results, or allow more sophisticated scenarios, such as the setting of alarms warning of low-blood sugar.

In the methods and medical uses of the present invention, the hypoglycaemia may be insulin-induced hypoglycaemia, for example following insulin administration after consumption of food by a patient, or hypoglycaemia induced by exercise, stress or illness. The glucagon dose for treatment of mild or moderate hypoglycaemia is generally administered to patients in an open loop setting. However, the patients may be treated with insulin in an open loop setting, a single hormone closed-loop setting (single hormone artificial/bionic pancreas (AP)), or a hybrid open-loop setting.

The methods and medical uses of the present invention may be adapted for use in which the glucagon is human native glucagon or else is a glucagon analogue, for example a glucagon analogue as set out in the detailed description below. For example, for a glucagon analogue the PK/PD parameters of the models might differ from those used for the human native glucagon, but the approaches described herein could be adapted by the skilled person employing those different parameters. Thus, data to inform the models must exist, then new simulations to determine the optimal bolus can be carried out. In preferred embodiments, the glucagon is human glucagon having the amino acid sequence Hy-HSQGTFTSDYSKYLDSRRAQDFVQWLMNT-OH or pharmaceutically acceptable salts and/or solvates thereof, or is a glucagon analogue having the amino acid sequences HSQGTFTSDYSKYLD-Aib-ARAEEFVKWLEST or HSQGTFTSDYSKYLD-Aib-ARAESFVKWLEST, or pharmaceutically acceptable salts and/or solvates thereof.

Conveniently, the optimum glucagon dose determined by the present invention is for administration as a bolus dose, for example for administration by subcutaneous injection or intramuscular injection. The size of the glucagon dose will generally be in the range between 25 μg and 1000 μg inclusive, or between 50 μg and 750 μg inclusive, or between 75 μg and 500 μg inclusive, or between 100 μg and 500 μg inclusive or between 125 μg and 500 μg inclusive. Thus the glucagon dose may be a dose of 25 μg, 50 μg, 75 μg, 100 μg, 125 μg, 150 μg, 175 μg, 200 μg, 250 μg, 300 μg, 400 μg, 500 μg or 1000 μg of glucagon. The medical uses and methods of the present invention may therefore be used for selecting the most appropriate dose of a glucagon for administration to a patient, e.g. from a group of two, three, four or five or more possible glucagon doses, having regard to aims and/or criteria for the treatment described herein.

In a further aspect, the present invention provides a bolus calculator for determining a dose for a glucagon bolus for administration to a patient with diabetes for treating mild or moderate hypoglycaemia, wherein the bolus calculator is configured to calculate the optimum dose by:
(a) determining an ambient insulin level for the patient, wherein the ambient insulin level is directly measured by a blood sample, measured by an insulin sensor and/or approximated by active insulin on board;
(bi) using the ambient insulin level to determine the dose for the glucagon bolus to treat mild or moderate hypoglycaemia while reducing the risk of, or avoiding, rebound hyperglycaemia according to treatment criteria (1) to increase plasma glucose (PG)≥5 mmol/l, (2) to have a peak plasma glucose (PG)≤10 mmol/l, and (3) to keep plasma glucose (PG)≥3.9 mmol/l for 120 min after the glucagon bolus;
(bii) using pharmacokinetic/pharmacodynamics (PK/PD) models for simulation of a virtual patient population of diabetes patients receiving the glucagon bolus to correct insulin-induced mild or moderate hypoglycaemia, wherein the dose of the glucagon bolus is the lowest glucagon dose yielding the maximal weighted success rate in the population calculated according to said treatment criteria; and
(c) selecting the lowest dose for the glucagon bolus that provides the maximal weighted success rate according to the treatment criteria.

In some embodiments, the bolus calculator takes account of insulin injections, carbohydrate intake and glucagon injections to account for side effects, treatment success, and IOB, thereby improving the prevention and treatment of hypoglycaemia, leading to improved glucose control.

Embodiments of the present invention will now be described by way of example and not limitation with reference to the accompanying figures. However, various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

### BRIEF DESCRIPTION OF THE FIGURES

FIG. 1: Schematic description of study design and treatment assessment. In seven virtual patients, 1 of 10 boluses of subcutaneous insulin was administered (t=−x) to decrease PG from 7.0 mmol/l to below 3.9 mmol/l. The insulin bolus size had to achieve predefined insulin levels when PG was 3.9 mmol/l. When PG reached 3.9 mmol/l (t=0), 1 of 17 subcutaneous glucagon boluses was administered. Treatment success of each glucagon dose was assessed on whether following peak PG was within 5 mmol/l (Green middle line: treatment limit) and 10 mmol/l (Blue top line: Hyperglycaemia limit), and whether PG 120 min after the glucagon bolus was above 3.9 mmol/l (Red other lines: Hypoglycaemia limit).

FIG. 2: Proportion of patients achieving treatment criteria as a function of glucagon dose, stratified by actual to baseline serum insulin concentrations. Treatment criteria were achieved if glucagon could increase PG to a peak above 5 mmol/l (Green line, crosses) and below 10 mmol/l (Blue line, open squares), and keep PG above 3.9 mmol/l for 120 min after the glucagon bolus (Red lines, open diamonds). The optimum glucagon dose for each serum insulin level (Black vertical line) was chosen as the lowest dose yielding the maximal weighted success rate of the three treatments criteria.

FIG. 3: Proportions of patients achieving treatment criteria as a function of glucagon dose stratified by insulin on board. Treatment criteria were achieved if glucagon could increase PG to a peak above 5 mmol/l (Green line, crosses) and below 10 mmol/l (Blue line, open squares), and keep PG above 3.9 mmol/l for 120 min after the glucagon bolus (Red lines, open diamonds). The optimum glucagon dose for each insulin on board (Black vertical line) was chosen as the lowest dose yielding the maximal weighted success rate of the three treatment criteria.

FIG. 4: Proportion of patients achieving treatment criteria as a function of glucagon dose and stratified by the percentage of insulin on board to the total daily insulin dose. Treatment criteria were achieved if glucagon could increase PG to a peak above 5 mmol/l (Green line, crosses) and below 10 mmol/l (Blue line, open squares), and keep PG above 3.9 mmol/l for 120 min after the glucagon bolus (Red lines, open diamonds). The optimum glucagon dose for each percentages of insulin on board (Black vertical line) was chosen as the lowest dose yielding the maximal weighted success rate of the three treatment criteria.

FIG. 5: Optimum glucagon dose as a function of ambient insulin levels stratified by actual to baseline serum insulin concentration (upper panel), insulin on board (middle panel), and percentage of insulin on board to total daily insulin dose (lower panel). Virtual patients performed 170 experiments per panel to obtain predefined ratios of insulin and glucagon at PG level of 3.9 mmol/l. The optimum glucagon dose to restore plasma glucose for each insulin level was chosen as the lowest glucagon dose yielding the maximal weighted success rate of the three treatment criteria 1) to increase PG above 5 mmol/l, 2) to have a peak PG below 10 mmol/l, and 3) to keep PG above 3.9 mmol/l for 120 min after the glucagon bolus.

FIG. 6: Patients will complete four study visits at the research facility in random order. On each visit, s.c. insulin bolus equal to 20% of patient's total daily insulin dose (IOB/TDD=20%) will be given in a fasting state in the morning, while a variable iv glucose infusion rate is given to maintain target PG level of 4.0 mmol/l until IOB/TDD achieves the predefined levels of 1, 3, 6 or 10%. The IOB follows a linear decay that will be zero depending on patient's insulin action time (IAT), which is close to reality. Once the predefined IOB/TDD level is reached, the glucose infusion rate is stopped and s.c. 100 μg glucagon is injected. PG levels will be monitored for another 180 min.

### DETAILED DESCRIPTION OF THE INVENTION

### Definitions

Unless specified otherwise, the following definitions are provided for specific terms, which are used herein.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats).

The glucagon and glucagon analogues (and pharmaceutically acceptable salts or solvates thereof) used in accordance with the present may be useful in the treatment or prevention of hypoglycaemia in patients with diabetes (“diabetic patients”), optionally used in combination with one or more additional therapeutically active substances. The present invention may be used in the treatment of hypoglycaemia in conscious diabetic patients, and particularly in the treatment of mild or moderate hypoglycaemia. Thus, the present invention may be used for the treatment of patients having hypoglycaemia who are conscious, as opposed to the treatment of severe hypoglycaemia where patients are unconscious. In moderate hypoglycaemia, patients may have a plasma glucose level between 3.0 and 3.9 mmol/l. The diabetic patients may have type 1 diabetes, type 2 diabetes or diabetes as a result of being pancreatectomized. The present invention is particularly useful for the treatment of mild or moderate hypoglycaemia in type 1 diabetes patients. Generally, the patients will be human patients and may be children or adults.

One aim of the present invention is to treat hypoglycaemia while reducing the risk of, or avoiding, rebound hyperglycaemia.

In the methods and medical uses of the present invention, “determining an ambient insulin level” for a patient includes measuring or approximating the concentration or amount of active insulin in the body, for example where the ambient insulin level is directly measured by a blood sample, is measured by an insulin sensor (e.g. within the subcutaneous (s.c.) compartment) and/or is approximated by active insulin on board

In the methods and medical uses of the present invention, using “a virtual patient population of diabetes patients” means using model equations and corresponding parameter estimates determined on a subject basis from clinical data to perform in silico experiments.

In the methods and medical uses of the present invention, the step of calculating the maximal weighted success rate uses a weighted harmonic mean (H) according to the formula:

$$H = \frac{1}{\frac{0.4}{{S}_{PG \geq 5}} + \frac{0.4}{{S}_{PG \leq 10}} + \frac{0.2}{{S}_{{PG}_{120} \geq 3.9}}}$$

and selecting the optimum dose comprises selecting the lowest glucagon dose with the highest H-value.

In the methods and medical uses of the present invention, reference to the use of “pharmacokinetic/pharmacodynamics (PK/PD) models” for simulation of a virtual patient population of diabetes patients includes the use of the PK/PD model described in detail in Wendt et al., Journal of Diabetes Science and Technology, 1-11, 2017, https://doi.or/10.1177/1932296817693254, the contents of which are expressly incorporated by reference in its entirety. The model employs the following PK/PD equations and parameters.

Insulin PK Model:

$$\frac{{dX}_{1} ( t )}{dt} = {u}_{I} ( t ) - \frac{{X}_{1} ( t )}{{t}_{max}}$$

$$\frac{{dX}_{2} ( t )}{dt} = \frac{{X}_{1} ( t )}{{t}_{max}} - \frac{{X}_{2} ( t )}{{t}_{max}}$$

$$I ( t ) = \frac{1}{{t}_{max}} \frac{{X}_{2} ( t )}{W \cdot {Cl}_{F , I}} {10}^{6} + {I}_{b}$$

TABLE 1
Summary of insulin PK model parameters for simulation with range
of means and 95% confidence interval (CI) or mean and 95% CI.

| | I_{b} | | tₘₐₓ | | CI_{F, I} | |
|---|---|---|---|---|---|---|
| Patient | [mU/l] | | [min] | | [ml/kg/min] | |
| 1 | 6.6-7.8 | (6.0-8.3) | 57.6 | (50.9-64.3) | 18.9 | (17.3-20.6) |
| 2 | 10.0-11.2 | (9.1-12.0) | 57.3 | (48.8-65.9) | 18.5 | (16.1-21.2) |
| 3 | 10.3-13.4 | (9.7-14.0) | 40.8 | (37.6-44.0) | 14.8 | (13.6-16.1) |
| 4 | 7.8-9.4 | (7.4-9.9) | 67.9 | (63.5-72.2) | 17.4 | (16.6-18.3) |
| 5 | 5.2-8.2 | (4.8-8.8) | 48.5 | (44.7-52.4) | 17.3 | (15.7-19.0) |
| 6 | 3.0-8.5 | (2.3-9.4) | 46.5 | (41.7-51.3) | 24.6 | (22.9-26.3) |
| 7 | 16.8-22.6 | (15.6-23.6) | 68.5 | (60.6-76.4) | 23.7 | (21.3-26.4) |
| 8 | 4.7-9.1 | (4.4-9.6) | 55.4 | (49.6-61.2) | 26.8 | (24.8-29.0) |

Glucagon PK Model:

$$\frac{{dZ}_{1} ( t )}{dt} = {u}_{C} ( t ) - {k}_{1} {Z}_{1} ( t )$$

$$\frac{{dZ}_{2} ( t )}{dt} = {k}_{1} {Z}_{1} ( t ) - {k}_{2} {Z}_{2} ( t )$$

$$C ( t ) = \frac{{k}_{2} {Z}_{2} ( t )}{W \cdot {Cl}_{F , C}} + {C}_{b}$$

TABLE 2
Summary of glucagon PK model parameters
for simulation with mean and 95% CI.

| | C_{b} | k₁ | k₂ | CI_{F, C} |
|---|---|---|---|---|
| Patient | [pg/ml] | [min⁻¹] | [min⁻¹] | [ml/kg/min] |
| 1 | 10.7 | 0.042 | 0.14 | 94 |
| | (9.4-12.0) | (0.036-0.048) | (0.10-0.22) | (83-105) |
| 2 | 7.6 | 0.056 | 0.26 | 106 |
| | (6.9-8.3) | (0.052-0.062) | (0.18-0.38) | (96-116) |
| 3 | 7.6 | 0.022 | 0.10 | 114 |
| | (5.9-9.3) | (0.018-0.028) | (0.06-0.17) | (96-132) |
| 4 | 10.9 | 0.058 | 0.058 | 159 |
| | (9.2-12.6) | (0.011-0.313) | (NA) | (133-184) |
| 5 | 8.7 | 0.038 | 0.19 | 200 |
| | (7.7-9.8) | (0.032-0.044) | (0.13-0.29) | (176-223) |
| 6 | 8.9 | 0.035 | 0.28 | 125 |
| | (7.8-10.0) | (0.031-0.040) | (0.19-0.41) | (111-138) |
| 7 | 11.6 | 0.035 | 0.25 | 136 |
| | (10.1-13.0) | (0.030-0.041) | (0.16-0.39) | (120-152) |
| 8 | 19.0 | 0.052 | 0.090 | 91 |
| | (16.1-22.0) | (0.037-0.072) | (0.04-0.26) | (78-105) |

PD Model:

$$\frac{{dQ}_{1} ( t )}{dt} = - {F}_{01} - {F}_{R} - {S}_{T} {x}_{1} ( t ) {Q}_{1} ( t ) + {k}_{12} {Q}_{2} ( t ) + {G}_{GG} ( t ) + {G}_{GNG}$$

$$\frac{{dQ}_{2} ( t )}{dt} = {S}_{T} {x}_{1} ( t ) {Q}_{1} ( t ) - [ {k}_{12} + {S}_{D} {x}_{2} ( t ) ] {Q}_{2} ( t )$$

$${G}_{GG} ( t ) = \frac{1 - {S}_{E} {x}_{3} ( t )}{1 - {S}_{E} {I}_{b}} ( ( {E}_{max} - {G}_{GNG} ) \frac{C ( t )}{{C}_{E 50} + C ( t )} ) \\ G ( t ) = \frac{{Q}_{1} ( t )}{V} \\ \frac{{dx}_{1} ( t )}{dt} = {k}_{a 1} [ I ( t ) - {x}_{1} ( t ) ] \\ \frac{{dx}_{2} ( t )}{dt} = {k}_{a 2} [ I ( t ) - {x}_{2} ( t ) ] \\ \frac{{dx}_{3} ( t )}{dt} = {k}_{a 3} [ I ( t ) - {x}_{3} ( t ) ]$$

TABLE 3
Summary of PD model parameters for simulation with mean and 95% CI.

| | C_{E50} | Eₘₐₓ | F₀₁ | | | | | S_{D}*10⁻⁴ | | S_{T}*10⁻⁴ |
|---|---|---|---|---|---|---|---|---|---|---|
| | [pg/ | [μmol/kg/ | [μmol/kg/ | k₁₂*10⁻⁴ | kₐ₁*10⁻⁴ | kₐ₂*10⁻⁴ | kₐ₃*10⁻⁴ | [min⁻¹/ | S_{E}*10⁻⁴ | [min⁻¹/ |
| ID | ml] | min] | min] | [min⁻¹] | [min⁻¹] | [min⁻¹] | [min⁻¹] | (mU/l)] | [(mU/l)⁻¹] | (mU/l)] |
| 1 | 436 | 56.4 | 14.2 | 244 | 16 | 522 | 215 | 1.5 | 155 | 23 |
| | (355- | (51.1- | (12.9- | (181- | (7- | (221- | (59- | (0.6- | (83- | (16- |
| | 517) | 61.8) | 15.5) | 330) | 35) | 1233) | 778) | 3.3) | 289) | 31) |
| 2 | 405 | 67.4 | 13.8 | 285 | 15 | 495 | 231 | 1.2 | 334 | 19 |
| | (339- | (59.3- | (12.8- | (223- | (7- | (236- | (137- | (0.6- | (232- | (15- |
| | 471) | 75.5) | 14.7) | 363) | 35) | 1039) | 389) | 2.3) | 481) | 25) |
| 3 | 401 | 57.4 | 15.5 | 397 | 18 | 548 | 327 | 1.4 | 237 | 25 |
| | (327- | (49.8- | (14.2- | (277- | (8- | (268- | (168- | (0.7- | (183- | (17- |
| | 475) | 65.0) | 16.8) | 568) | 42) | 1121) | 638) | 2.5) | 308) | 36) |
| 4 | 285 | 84.4 | 12.8 | 213 | 18 | 437 | 68 | 2.0 | 415 | 18 |
| | (226- | (73.9- | (11.3- | (157- | (9- | (183- | (42- | (1.0- | (347- | (13- |
| | 344) | 94.8) | 14.4) | 289) | 36) | 1044) | 113) | 3.8) | 496) | 25) |
| 5 | 339 | 65.4 | 12.0 | 281 | 15 | 517 | 235 | 1.1 | 229 | 31 |
| | (251- | (53.8- | (10.6- | (194- | (7- | (223- | (95- | (0.4- | (127- | (20- |
| | 427) | 77.1) | 13.5) | 406) | 32) | 1201) | 586) | 2.6) | 415) | 47) |
| 6 | 424 | 60.1 | 13.1 | 238 | 10 | 353 | 74 | 2.6 | 404 | 21 |
| | (333- | (46.3- | (11.7- | (172- | (4- | (102- | (23- | (1.1- | (185- | (14- |
| | 515) | 74.0) | 14.5) | 330) | 22) | 1221) | 232) | 6.2) | 882) | 32) |
| 7 | 141 | 78.0 | 14.2 | 358 | 49 | 624 | 178 | 4.4 | 140 | 21 |
| | (96- | (68.9- | (12.2- | (252- | (23- | (319- | (69- | (3.2- | (99- | (16- |
| | 187) | 87.1) | 16.1) | 509) | 105) | 1221) | 459) | 6.0) | 199) | 29) |
| 8 | 307 | 75.3 | 13.4 | 289 | 37 | 518 | 154 | 4.2 | 463 | 29 |
| | (228- | (61.5- | (11.4- | (197- | (18- | (203- | (68- | (2.8- | (377- | (20- |
| | 386) | 89.1) | 15.4) | 424) | 75) | 1324) | 348) | 6.5) | 569) | 42) |

Furthermore:

G_{GNG} is fixed at 6 μmol/kg/minute (Nuttall et al., Regulation of hepatic glucose production and the role of gluconeogenesis in humans: is the rate of gluconeogenesis constant?

Diabetes Metab Res Rev. 2008; 24: 438-458).

F₀₁ is constant when plasma glucose concentration exceeds 81 mg/dl [30]. Otherwise it follows:

$$\frac{{F}_{01} G ( t )}{4.5}$$

F_{R} is zero when plasma glucose concentrations do not exceed 162 mg/dl (Hovorka et al., Nonlinear model predictive control of glucose concentration in subjects with type 1 diabetes. Physiol. Meas. 2004; 25: 905-920.). Otherwise it follows:

0.003(G(t)−9)V

V is fixed at 160 ml/kg (Hovorka et al., Partitioning glucose distribution/transport, disposal, and endogenous production during IVGTT. Am J Physiol Endocrinol Metab. 2002; 282: E992-E1007).

TABLE 4
Interpretation of insulin PK (top rows), glucagon PK (middle rows)
and glucose PD (bottom rows) model parameters and their units.

| Parameter | Unit | Interpretation |
|---|---|---|
| X₁(t) | U | insulin mass due to exogenous dosing, |
| | | in SC tissue |
| X₂(t) | U | insulin mass due to exogenous dosing, |
| | | in serum |
| u_{I}(t) | U/minute | insulin dose |
| tₘₐₓ | minutes | time from dose to maximum serum |
| | | concentration |
| W | kg | body weight |
| CI_{F, I} | ml/kg/minute | apparent insulin clearance |
| I_{b} | mU/l | steady state insulin concentration |
| I(t) | mU/l | insulin concentration in serum |
| Z₁(t) | pg | glucagon mass due to exogenous dosing, |
| | | in SC tissue |
| Z₂(t) | pg | glucagon mass due to exogenous dosing, |
| | | in plasma |
| u_{C}(t) | pg/minute | glucagon dose |
| k₁ | minute⁻¹ | absorption rate constant |
| k₂ | minute⁻¹ | elimination rate constant |
| CI_{F, C} | ml/kg/minute | apparent glucagon clearance |
| C_{b} | pg/ml | steady state glucagon concentration |
| C(t) | pg/ml | glucagon concentration in plasma |
| Q₁(t) | μmol/kg | glucose mass per W in the |
| | | accessible compartment |
| Q₂(t) | μmol/kg | glucose mass per W in the non- |
| | | accessible compartment |
| x₁(t) | mU/l | remote effects of insulin on |
| | | glucose transport |
| x₂(t) | mU/l | remote effects of insulin on |
| | | glucose disposal |
| x₃(t) | mU/l | remote effects of insulin on |
| | | glycogenolysis |
| G(t) | mmol/l | glucose concentration in plasma |
| G_{GG}(t) | μmol/kg/minute | glucose production due to |
| | | glycogenolysis |
| G_{GNG} | μmol/kg/minute | glucose production due to |
| | | gluconeogenesis |
| F₀₁ | μmol/kg/minute | insulin independent glucose flux |
| F_{R} | μmol/kg/minute | renal glucose clearance |
| S_{T} | minute⁻¹/(mU/l) | insulin sensitivity of glucose transport |
| S_{D} | minute⁻¹/(mU/l) | insulin sensitivity of glucose disposal |
| S_{E} | l/mU | insulin sensitivity on glycogenolysis |
| k₁₂ | minute⁻¹ | transfer rate constant from the non- |
| | | accessible to the accessible compartment |
| kₐ₁ | minute⁻¹ | insulin deactivation rate constant |
| kₐ₂ | minute⁻¹ | insulin deactivation rate constant |
| kₐ₃ | minute⁻¹ | insulin deactivation rate constant |
| Eₘₐₓ | μmol/kg/minute | maximum EGP at basal insulin |
| | | concentration |
| C_{E50} | pg/ml | glucagon concentration yielding |
| | | half of maximum EGP |
| V | ml/kg | glucose volume of distribution |

PG: abbreviation for plasma glucose concentration.
TDD: abbreviation for total daily insulin dosage.

In addition to the explanations of the meanings of certain terms or expressions employed in the present specification that are provided in the above, the following definitions/explanations also apply:

The term “pharmaceutically acceptable carrier” includes any of the standard pharmaceutical carriers or diluents, such as those used in compositions or formulations suitable for oral, pulmonary, rectal, nasal, topical, subcutaneous, intramuscular, intravenous, intraperitoneal, intradermal, transdermal or vaginal administration. Pharmaceutically acceptable carriers for therapeutic use are well known in the pharmaceutical art, and are described, for example, in Remington's Pharmaceutical Sciences, Mack Publishing Co. (A. R. Gennaro edit. 1985). Liquid compositions often employ unbuffered or buffered aqueous solutions as carriers. For example, sterile saline or phosphate-buffered saline (PBS) at slightly acidic, slightly alkaline or physiological pH may be used. Relevant pH-buffering agents (some of which have already been mentioned above in connection with pharmaceutical compositions) include phosphates, citrate, acetate, tris(hydroxymethyl)aminomethane (TRIS), N-tris(hydroxymethyl)methyl-3-aminopropane-sulfonic acid (TAPS), ammonium bicarbonate, diethanolamine, histidine (which is often a preferred buffer), arginine and lysine, as well as mixtures thereof. The term further encompasses any agents listed in the US Pharmacopeia for use in animals or humans.

The term “pharmaceutically acceptable salt” in the context of the invention refers to a salt that is not harmful to the patient or subject to be treated therewith. Such salts are in general acid addition salts or basic salts. Acid addition salts include salts of inorganic acids and salts of organic acids. Non-limiting examples of suitable acid addition salts include hydrochloride salts, phosphate salts, formate salts, acetate salts, trifluoroacetate salts and citrate salts. Examples of basic salts include salts where the cation is selected from alkali metal ions, such as sodium and potassium, alkaline earth metal ions, such as calcium, as well as substituted ammonium ions, e.g. of the type NR(R′)₃⁺, where R and R′ independently designate optionally substituted C₁₋₆alkyl, optionally substituted C₂₋₆alkenyl, optionally substituted aryl, or optionally substituted heteroaryl. Other examples of pharmaceutically acceptable salts are described in Remington's Pharmaceutical Sciences, 17th edition. Ed. Alfonso R. Gennaro (Ed.), Mack Publishing Company, Easton, Pa., U.S.A., 1985 and more recent editions, and in the Encyclopaedia of Pharmaceutical Technology.

The term “solvate” in the context of the present invention refers to a complex of defined stoichiometry formed by a solute (in casu a compound, or a pharmaceutically acceptable salt thereof, of the present invention) and a solvent. Relevant solvents (particularly in the case of pharmaceutically acceptable solvates) include, but are not limited to, water, ethanol and acetic acid. Solvates in which the solvent molecule in question is water are generally referred to as “hydrates”.

The terms “therapeutically effective amount” and “therapeutically effective dose” as employed in the context of the present invention (notably in the context of a compound of the invention) refer to an amount or a dose sufficient to cure, alleviate, partially arrest or otherwise promote the cure or healing of a given condition (disorder, disease) or injury and, preferably, complications arising therefrom. An amount or dose effective for a particular purpose will depend on the severity of the condition or injury as well as on the body weight and general state of the subject or patient to be treated. Determination of an amount or dose that is appropriate is within the skills of a trained physician (or veterinarian) of ordinary skill.

The term “treatment” (as well as “treating” and other grammatical variants thereof) as employed in the context of the invention refers to an approach for obtaining beneficial or desired clinical results. For the purposes of the present invention, beneficial or desired clinical results include, but are not limited to, alleviation of symptoms, diminishment of extent of disease, stabilization of (i.e. not worsening of) state of disease, delay or slowing of disease progression, amelioration or palliation of disease state, and remission (whether partial or total), whether detectable or undetectable. “Treatment” may also refer to prolongation of survival compared to expected survival in the absence of treatment.

“Treatment” is an intervention performed with the intention of preventing the development of, or altering the pathology of, a disorder. Accordingly, “treatment” refers both to therapeutic treatment and to prophylactic or preventative measures. As used in the context of prophylactic or preventative measures, the compound need not completely prevent the development of the disease or disorder. Those in need of treatment include those already suffering from the disorder, as well as those in which development of the disorder is to be prevented. “Treatment” also means inhibition or reduction of an increase in pathology or symptoms (e.g. weight gain or hypoglycaemia) compared to the absence of treatment, and is not necessarily meant to imply complete cessation of the relevant condition.

The term “agonist” as employed in the context of the invention refers to a substance (ligand) that activates the receptor type in question.

Throughout the present specification, the conventional one-letter and three-letter codes for naturally occurring amino acids are used. Unless otherwise indicated, reference is made to the L-isomeric forms of the amino acids referred to herein.

Dab(Ac): 4-N-Acetyl-2,4-diaminobutyric acid, (2S)-4-(Acetylamino)-2-aminobutanoic acid or 4-(acetylamino)-2-aminobutanoic acid (L-form).

Dap(Ac): 3-N-Acetyl-2,3-diaminopropionic acid or 3-(acetylamino)-2-aminopropanoic acid (L-form)

Gln(Me): N-6-methyl-L-glutamine

N-Me-Tyr: Tyrosine which is methylated at the α-nitrogen

N-Me-DTyr: D-Tyrosine which is methylated at the α-nitrogen

N-Me-Ser: Serine which is methylated at the α-nitrogen

N-Me-DSer: D-Serine which is methylated at the α-nitrogen

Aib: α-aminoisobutyric acid

In the present invention, references to “a glucagon” includes the use of native glucagon (e.g. native human glucagon) and/or glucagon analogues. Native human glucagon is a 29 amino acid native human glucagon peptide that corresponds to amino acids 53 to 81 of pre-proglucagon and which has the sequence Hy-HSQGTFTSDYSKYLDSRRAQDFVQWLMNT-OH (SEQ ID NO: 1). A HCl salt of native glucagon is approved under the name “GlucaGen”.

Among sequences disclosed herein are sequences incorporating an “Hy-” moiety at the amino terminus (N-terminus) of the sequence, and either an “—OH” moiety or an “—NH₂” moiety at the carboxy terminus (C-terminus) of the sequence. In such cases, and unless otherwise indicated, an “Hy-” moiety at the N-terminus of the sequence in question indicates a hydrogen atom [i.e. R¹=hydrogen=Hy- in formulas I and Ia; corresponding to the presence of a free primary or secondary amino group at the N-terminus], while an “—OH” or an “—NH₂” moiety at the C-terminus of the sequence indicates a hydroxy group [e.g., R²=OH in formulas I and Ia; corresponding to the presence of a carboxy (COOH) group at the C-terminus] or an amino group [e.g., R²=NH₂ in formulas I and Ia; corresponding to the presence of an amido (CONH₂) group at the C-terminus], respectively. In each sequence of the invention, a C-terminal “—OH” moiety may be substituted for a C-terminal “—NH₂” moiety, and vice-versa.

In addition to the use of native human glucagon, the present invention may be adapted to use glucagon analogues, for example the stable glucagon analogues suitable for use in a liquid formulation, the synthesis and uses of which are disclosed in WO 2014/016300, WO 2012/130866, WO 2013/041678 and WO 2015/124612, all of which are hereby expressly incorporated by reference in their entirety. These analogues are described below and include the glucagon analogues HSQGTFTSDYSKYLD-Aib-ARAEEFVKWLEST (SEQ ID NO: 22, WO 2014/016300) and HSQGTFTSDYSKYLD-Aib-ARAESFVKWLEST (SEQ ID NO: 16, WO 2014/016300), or a salt or solvate thereof.

Glucagon and glucagon analogues help to maintain the level of glucose in the blood by binding to glucagon receptors on hepatocytes, causing the liver to release glucose—stored in the form of glycogen—through glycogenolysis. As these stores become depleted, glucagon also stimulates the liver to synthesize additional glucose by gluconeogenesis.

This glucose is released into the bloodstream, preventing the development of hypoglycaemia.

Some embodiments of the present invention relate to compounds having the formula I:

R¹—Z—R² (I)

or a pharmaceutically acceptable salt or solvate thereof;

wherein

R¹ is hydrogen-, C₁₋₄ alkyl, acetyl, formyl, benzoyl or trifluoroacetyl;

R² is —OH or —NH₂; and

Z is an amino acid sequence deriving from the sequence of formula Ia:

His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Glu-Asn-Thr (Ia)

and further comprising at least tour amino acid substitutions or deletions that are only at sequence positions (designated by an X) selected from 2, 3, 4, 9, 10, 15, 16, 17, 20, 21, 24, 28 and 29, as follows:

X2 is selected from Aib and Ala;

X3 is selected from His, Pro, Dab(Ac), Dap(Ac) and Gln(Me);

X4 is DAla;

X9 is Glu;

X10 is selected from Val, Leu N-Me-Tyr and N-Me-DTyr;

X15 is Glu;

X16 is selected from Aib, Lys, Glu, Leu, Val, DVal, Phe, His, Arg, Pro, DPro, N-Me-Ser and N-Me-DSer;

X17 is selected from Ala and Ser;

X20 is selected from Glu and Lys;

X21 is selected from Glu, Lys and Ser;

X24 is selected from Lys, Ser, Glu and Ala;

X25 is selected from Arg, Lys, His, lie, Leu, Ala, Met, Cys, Asn, Val, Ser, Glu, Asp, Gin, Thr and (p)Tyr;

X28 is selected from Ser, Lys, and Glu, or is absent;

X29 is selected from Ser and Ala, or is absent; optionally with the proviso that Z is not selected from:

HSQGTFTSDYSKYLDSARAEDFVKWLEST;
and
HSQGTFTSDYSKYLESRRAKEFVEWLEST.

Some embodiments of the present invention relate to compounds having the formula I:

R¹—Z—R² (I)

or a pharmaceutically acceptable salt or solvate thereof;

wherein

R¹ is hydrogen-, C₁₋₄ alkyl, acetyl, formyl, benzoyl or trifluoroacetyl;

R² is —OH or —NH₂; and

Z is an amino acid sequence deriving from the sequence of formula Ia:

His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Glu-Asn-Thr (Ia)

and further comprising at least four amino acid substitutions or deletions that are only at sequence positions (designated by an X) selected from 2, 3, 9, 10, 15, 16, 17, 20, 21, 24, 28 and 29, as follows:

X2 is selected from Aib and Ala;

X3 is selected from His and Pro;

X9 is Glu;

X10 is selected from N-Me-Tyr and N-Me-DTyr;

X15 is Glu;

X16 is selected from Aib, Lys, Glu, Leu, Val, DVal, Phe, His, Arg, Pro, DPro, N-Me-Ser and N-Me-DSer;

X17 is selected from Ala and Ser;

X20 is selected from Glu and Lys;

X21 is selected from Glu, Lys and Ser;

X24 is selected from Lys, Ser, Glu and Ala;

X25 is selected from Arg, Lys, His, lie, Leu, Ala, Met, Cys, Asn, Val, Ser, Glu, Asp, Gin, Thr and (p)Tyr;

X28 is selected from Ser and Lys, or is absent;

X29 is selected from Ser and Ala, or is absent;

optionally with the proviso that Z is not selected from:

HSQGTFTSDYSKYLDSARAEDFVKWLEST;
and
HSQGTFTSDYSKYLESRRAKEFVEWLEST.

In some embodiments, the at least four amino acid substitutions or deletions at amino acid sequence positions (designated by an X) selected from 2, 3, 4, 9, 10, 15, 16, 17, 20, 21, 24, 28 and 29 of the compound of formula I are as follows:

X2 is selected from Aib and Ala;

X3 is selected from His and Pro, Dab(Ac), Dap(Ac) and Gln(Me);

X4 is DAla;

X9 is Glu;

X10 is selected from Val, Leu, N-Me-Tyr and N-Me-DTyr;

X15 is Glu;

X16 is selected from Aib, Lys, Glu, Leu, Val, Phe, His and Arg;

X17 is selected from Ala and Ser;

X20 is selected from Glu and Lys;

X21 is selected from Glu, Lys and Ser;

X24 is selected from Lys, Ser, Glu and Ala;

X28 is selected from Ser, Glu and Lys, or is absent;

X29 is selected from Ser and Ala, or is absent.

In some embodiments, the at least four amino acid substitutions or deletions are at amino acid sequence positions (designated by an X) selected from 2, 3, 4, 10, 15, 16, 17, 20, 21, 24, 28 and 29 of the compound of formula I, and are as follows:

X2 is Ala;

X3 is Dab(Ac) and Gln(Me);

X4 is DAla;

X10 is selected from Leu and Val;

X15 is Glu;

X16 is selected from Aib, Lys, Glu, Leu, and Val;

X17 is Ala;

X20 is selected from Glu and Lys;

X21 is selected from Glu and Ser;

X24 is selected from Lys, Ser and Glu;

X28 is selected from Ser, Glu and Lys;

X29 is Ala, or is absent. In some embodiments, X3 is selected from Dab(Ac) and Gln(Me).

In some embodiments, the at least four amino acid substitutions or deletions are at amino acid sequence positions (designated by an X) selected from 2, 3, 4, 16, 17, 20, 21, 24, 28 and 29 of the compound of formula I, and are as follows:

X2 is Ala;

X3 is Dab(Ac), Dap(Ac), Gln(Me) or His;

X4 is DAla;

X16 is selected from Aib, Lys, Glu;

X17 is Ala;

X20 is selected from Glu and Lys;

X21 is selected from Glu and Ser;

X24 is selected from Lys, Ser and Glu;

X28 is selected from Ser, Glu and Lys;

X29 is Ala, or is absent.

In some embodiments, X17 is Ala.

In some embodiments, X25 is selected from Arg, His or Lys. In some embodiments, the compounds of the invention may comprise substitutions in position 25, such as those referred to in WO 2011/117417, which is incorporated herein by reference. However, such substitutions at position 25 are not necessary in the present invention to obtain enhanced physical stability of the glucagon analogues.

In some embodiments, X27 is selected from Ser, Lys, Glu, and Asp. In some embodiments, X27 is selected from: Glu and Asp. In some embodiments, X27 is Glu.

In some embodiments, X28 and/or X29 may be amino acid residues other than those disclosed above. In some embodiments, the substitution may be a hydrophilic substitution (e.g., Arg, Lys, Asn, His, Gin, Asp, Ser, or Glu). In some embodiments, X28 and/or X29 may be selected from: Glu, Asp, Lys, Arg, Ser, Leu, Ala and Gly. In some embodiments, X28 is Glu or Asp. In some embodiments, X29 is Glu or Asp. In some embodiments, X28 is Glu and X29 is Glu.

In some embodiments, X17 is Ala and X27 is Glu. In some embodiments, X20 is Glu and X27 is Glu. In some embodiments, X17 is Ala, X20 is Glu, and X27 is Glu. In some embodiments, X16 is Aib and X27 is Glu. In some embodiments, X16 is Aib, X21 is Ser, and X27 is Glu. In some embodiments, X16 is Aib, X21 is Ser, X27 is Glu, and X28 is Ser. In addition to the possibility of substitution of the amino acid residue at position 3 (X3) in formula Ia with an amino acid residue selected from His, Pro, Dab(Ac) and Gln(Me), position 3 may also be substituted with an analogue of glutamine, which will typically be an unnatural amino acid (i.e. one not naturally occurring in mammalian proteins) such as Dap(Ac) [i.e. X3=Dap(Ac)]. Nonetheless, in all of the definitions provided herein, the invention further encompasses compounds defined by the same generic formulae but in which Dap(Ac) is not permitted at X3.

In some embodiments of compounds of the invention, Z is selected from the group consisting of:

(SEQ ID NO: 2)
HSQGTFTSDYSKYLDSARAESFVKWLEST
(SEQ ID NO: 3)
HSQGTFTSDYSKYLDSARAEDFVKWLEET
(SEQ ID NO: 4)
HSQGTFTSDYSKYLDKARAEDFVKWLEST
(SEQ ID NO: 5)
HSQGTFTSDYSKYLDSARAEDFVAWLEST
(SEQ ID NO: 6)
HSQGTFTSDYSKYLDEARAKDFVEWLEKT
(SEQ ID NO: 7)
HSQGTFTSDYSKYLDSARAEDFVEWLEST
(SEQ ID NO: 8)
HSQGTFTSDYSRYLESARAEDFVKWLEST
(SEQ ID NO: 9)
HSQGTFTSDYSKYLESARAEDFVKWLEST
(SEQ ID NO: 10)
HSQGTFTSDYSKYLDSARAEEFVKWLEST
(SEQ ID NO: 11)
HSQGTFTSDYSKYLDSARAEDFVSWLEST
SEQ ID NO: 12)
HSQGTFTSDLSKYLDSARAEDFVKWLEST
(SEQ ID NO: 13)
HSQGTFTSDYSKYLD-Aib-ARAEDFVKWLEST
(SEQ ID NO: 14)
HSQGTFTSDYSKYLDSARAEDFVKWLES
(SEQ ID NO: 15)
HSQGTFTSDYSKYLDEARAEDFVKWLEST
(SEQ ID NO: 16)
HSQGTFTSDYSKYLD-Aib-ARAESFVKWLEST
(SEQ ID NO: 17)
HSQGTFTSDYSKYLESARAESFVKWLEST
(SEQ ID NO: 18)
HSQGTFTSDYSKYLDLARAEDFVKWLEST
(SEQ ID NO: 19)
HSQGTFTSDYSKYLDKRRAEDFVSWLEST
(SEQ ID NO: 20)
HSQGTFTSDYSKYLDVARAESFVKWLEST
(SEQ ID NO: 21)
HAQGTFTSDYSKYLD-Aib-ARAESFVKWLEST
(SEQ ID NO: 22)
HSQGTFTSDYSKYLD-Aib-ARAEEFVKWLEST
(SEQ ID NO: 23)
HSQ-DAla-TFTSDYSKYLD-Aib-ARAESFVKWLEST
(SEQ ID NO: 24)
HSQGTFTSDVSKYLD-Aib-ARAESFVKWLEST
(SEQ ID NO: 25)
HS-[Dab(Ac)]-GTFTSDYSKYLD-Aib-ARAESFVKWLEST
(SEQ ID NO: 26)
HSQGTFTSDYSKYLD-Aib-RRAESFVKWLEST
(SEQ ID NO: 27)
HS-[Gln(Me)]-GTFTSDYSKYLD-Aib-ARAESFVKWLEST
(SEQ ID NO: 28)
HSQGTFTSDYSKYLDEARAKSFVEWLEKT
(SEQ ID NO: 29)
HSQGTFTSDYSKYLDEARAKSFVEWLEST
(SEQ ID NO: 30)
HSQGTFTSDYSKYLD-Aib-ARAKSFVEWLEKT
(SEQ ID NO: 31)
HSQGTFTSDYSKYLD-Aib-ARAESFVKWLESA
(SEQ ID NO: 32)
HSQGTFTSDYSKYLD-Aib-ARAESFVK1NLEST
(SEQ ID NO: 33)
HS-[Dab(Ac)]-GTFTSDYSKYLD-Aib-ARAESFVKWLEST
(SEQ ID NO: 34)
HSQGTFTSDYSKYLD-Aib-ARAEEFVSWLEKT
(SEQ ID NO: 35)
HSQGTFTSDYSKYLD-Aib-ARAEKFVEWLEST
(SEQ ID NO: 36)
HSQGTFTSDYSKYLD-Aib-ARAEEFVAWLEST
(SEQ ID NO: 37)
HSQGTFTSDYSKYLD-Aib-ARAEEFVKWLEET
(SEQ ID NO: 38)
HSQGTFTSDYSKYLE-Aib-ARAEEFVKWLEST
(SEQ ID NO: 39)
HSHGTFTSDYSKYLD-Aib-ARAEEFVKWLEST
(SEQ ID NO: 40)
HS-[Dab(Ac)]-GTFTSDYSKYLD-Aib-ARAEEFVKWLEST
and
(SEQ ID NO: 41)
HS-[Dap(Ac)]-GTFTSDYSKYLD-Aib-ARAEEFVKWLEST.

Specific compounds of the invention include:

Hy-HSQGTFTSDYSKYLDSARAESFVKWLEST-OH Compound 1;
Hy-HSQGTFTSDYSKYLDSARAEDFVKWLEET-OH Compound 2;
Hy-HSQGTFTSDYSKYLDKARAEDFVKWLEST-OH Compound 3;
Hy-HSQGTFTSDYSKYLDSARAEDFVAWLEST-OH Compound 4;
Hy-HSQGTFTSDYSKYLDEARAKDFVEWLEKT-OH Compound 5;
Hy-HSQGTFTSDYSKYLDSARAEDFVEWLEST-OH Compound 6;
Hy-HSQGTFTSDYSRYLESARAEDFVKWLEST-OH Compound 7;
Hy-HSQGTFTSDYSKYLESARAEDFVKWLEST-OH Compound 8;
Hy-HSQGTFTSDYSKYLDSARAEEFVKWLEST-OH Compound 9;
Hy-HSQGTFTSDYSKYLDSARAEDFVSWLEST-OH Compound 10;
Hy-HSQGTFTSDLSKYLDSARAEDFVKWLEST-OH Compound 11;
Hy-HSQGTFTSDYSKYLD-Aib-ARAEDFVKWLEST-OH Compound
12;
Hy-HSQGTFTSDYSKYLDSARAEDFVKWLES-OH Compound 13;
Hy-HSQGTFTSDYSKYLDEARAEDFVKWLEST-OH Compound 14;
Hy-HSQGTFTSDYSKYLD-Aib-ARAESFVKWLEST-OH Compound
15;
Hy-HSQGTFTSDYSKYLESARAESFVKWLEST-OH Compound 16;
Hy-HSQGTFTSDYSKYLDLARAEDFVKWLEST-OH Compound 17;
Hy-HSQGTFTSDYSKYLDKRRAEDFVSWLEST-OH Compound 18;
Hy-HSQGTFTSDYSKYLDVARAESFVKWLEST-OH Compound 19;
Hy-HAQGTFTSDYSKYLD-Aib-ARAESFVKWLEST-OH Compound
20;
Hy-HSQGTFTSDYSKYLD-Aib-ARAEEFVKWLEST-OH Compound
21;
Hy-HSQ-DAla-TFTSDYSKYLD-Aib-ARAESFVKWLEST-OH
Compound 22;
Hy-HSQGTFTSDVSKYLD-Aib-ARAESFVKWLEST-OH Compound
23;
Hy-HS-[Dab(Ac)]-GTFTSDYSKYLD-Aib-ARAESFVKWLEST-NH₂
Compound 24;
Hy-HSQGTFTSDYSKYLD-Aib-RRAESFVKWLEST-OH Compound
25;
Hy-HS-[Gln(Me)]-GTFTSDYSKYLD-Aib-ARAESFVKWLEST-OH
Compound 26;
Hy-HSQGTFTSDYSKYLDEARAKSFVEWLEKT-OH Compound
27;
Hy-HSQGTFTSDYSKYLDEARAKSFVEWLEST-OH Compound
28;
Hy-HSQGTFTSDYSKYLD-Aib-ARAKSFVEWLEKT-OH Compound
29;
Hy-HSQGTFTSDYSKYLD-Aib-ARAESFVKWLESA-OH Compound
30;
Hy-HSQGTFTSDYSKYLD-Aib-ARAESFVKWLEST-NH₂ Compound
31;
Hy-HS-[Dab(Ac)]-GTFTSDYSKYLD-Aib-ARAESFVKWLEST-OH
Compound 32;
Hy-HSQGTFTSDYSKYLD-Aib-ARAEEFVSWLEKT-OH Compound
33;
Hy-HSQGTFTSDYSKYLD-Aib-ARAEKFVEWLEST-OH Compound
34;
Hy-HSQGTFTSDYSKYLD-Aib-ARAEEFVAWLEST-OH Compound
35;
Hy-HSQGTFTSDYSKYLD-Aib-ARAEEFVKWLEET-OH Compound
36;
Hy-HSQGTFTSDYSKYLE-Aib-ARAEEFVKWLEST-OH Compound
37;
Hy-HSHGTFTSDYSKYLD-Aib-ARAEEFVKWLEST-NH₂ Compound
38;
Hy-HS-[Dab(Ac)]-GTFTSDYSKYLD-Aib-ARAEEFVKWLEST-OH
Compound 39;
Hy-HS-[Dab(Ac)]-GTFTSDYSKYLD-Aib-ARAEEFVKWLEST-NH₂
Compound 40;
Hy-HS-[Dap(Ac)]-GTFTSDYSKYLD-Aib-ARAEEFVKWLEST-NH₂
Compound 41;

and pharmaceutically acceptable salts and solvates thereof.

Compounds of the invention may have one or more intramolecular bridges within the peptide sequence. Each such bridge is formed between the side-chains of two amino acid residues in the sequence which are typically separated by three other amino acid residues (i.e. between a side-chain of amino acid A and a side-chain of amino acid A+4).

For example, such a bridge may be formed between the side-chains of amino acid residue pairs 12 and 16, 16 and 20, 20 and 24, or 24 and 28. The two side-chains in question may be linked to one another through ionic interactions, or via covalent bonds. Thus, such pairs of amino acid residues may for example contain oppositely charged side-chains capable of forming a salt bridge or resulting in an ionic interaction. In such cases, one of the amino acid residues in question may, for example, be Glu or Asp, while the other may, for example, be Lys or Arg. Pairing of Lys and Glu or Lys and Asp may also lead to formation of a lactam ring.

Pharmaceutical Compositions

In some embodiments, the present invention relates to pharmaceutical compositions comprising a compound (or a pharmaceutically acceptable salt or solvate thereof) of the invention and a pharmaceutically acceptable carrier. Such pharmaceutical compositions may be prepared by conventional techniques, e.g. as described in Remington: The Science and Practice of Pharmacy, 19^{th} edition, 1995.

Certain embodiments of liquid pharmaceutical compositions of the invention may comprise a compound of the invention present in a concentration from about 0.01 mg/ml to about 25 mg/ml, such as from about 1 mg/ml to about 10 mg/ml, e.g. from about 1 mg/ml to about 5 mg/ml. In some embodiments, the composition has a pH from 2.0 to 10.0. A pharmaceutical composition of the invention may further comprise a buffer system, preservative(s), isotonicity agent(s), chelating stabilizer(s) and/or surfactant(s). Particularly useful embodiments of liquid pharmaceutical compositions of the invention are aqueous compositions, i.e. compositions comprising water. Such compositions may be in the form of an aqueous solution or an aqueous suspension. Preferred embodiments of aqueous pharmaceutical compositions of the invention are aqueous solutions. In the context of the invention the term “aqueous composition” will normally refer to a composition comprising at least 50% by weight (50% w/w) of water. Likewise, the term “aqueous solution” will normally refer to a solution comprising at least 50% w/w of water, and the term “aqueous suspension” to a suspension comprising at least 50% w/w of water.

In some embodiments, a pharmaceutical composition of the invention comprises an aqueous solution of a compound (or a pharmaceutically acceptable salt or solvate thereof) of the invention present at a concentration of from 0.1 mg/ml or above, together with a buffer, the composition having a pH from about 2.0 to about 10.0, such as a pH from about 6.0 to about 8.5, e.g. from about 6.5 to about 8.5, such as from about 7.0 to about 8.5, or from about 6.5 to about 8.0.

In other embodiments of a pharmaceutical composition of the invention, the pH of the composition is a pH selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.8, 9.9, and 10.0. The pH of the composition may be at least 1 pH unit from (i.e., higher or lower than) the isoelectric point of the constituent compound of the invention, such as at least 2 pH units from (i.e., higher or lower than) the isoelectric point of the glucagon analogue compound of the invention.

In further embodiments of buffer-containing pharmaceutical compositions of the invention, the buffer or buffer substance is selected from the group consisting of: acetate buffers (e.g. sodium acetate), sodium carbonate, citrates (e.g. sodium citrate), glycylglycine, histidine, glycine, lysine, arginine, phosphates (e.g. chosen among sodium dihydrogen phosphate, disodium hydrogen phosphate and trisodium phosphate), TRIS (i.e., tris(hydroxymethyl)aminomethane), HEPES (i.e., 4-(2-hydroxyethyl)-1-piperazine-ethanesulfonic acid), BICINE (i.e., N,N-bis(2-hydroxyethyl)glycine), and TRICINE (i.e., N-[tris(hydroxymethyl)methyl]glycine), as well as succinate, malate, maleate, fumarate, tartrate, and aspartate buffers, and mixtures thereof.

In further embodiments of pharmaceutical compositions of the invention, the composition comprises a pharmaceutically acceptable preservative. Relevant preservatives include preservatives selected from the group consisting of: phenol, o-cresol, m-cresol, p-cresol, methyl p-hydroxybenzoate, ethyl p-hydroxybenzoate, propyl p-hydroxybenzoate, butyl p-hydroxybenzoate, 2-phenoxyethanol, 2-phenylethanol, benzyl alcohol, ethanol, chlorobutanol, thiomerosal, bronopol, benzoic acid, imidurea, chlorhexidine, sodium dehydroacetate, chlorocresol, benzethonium chloride, chlorphenesine [i.e. 3-(p-chlorphenoxy)propane-1,2-diol] and mixtures thereof. The preservative may be present in a concentration of from 0.1 mg/ml to 30 mg/ml, such as from 0.1 mg/ml to 20 mg/ml (e.g. from 0.1 mg/ml to 5 mg/ml, or from 5 mg/ml to 10 mg/ml, or from 10 mg/ml to 20 mg/ml) in the final liquid composition. The use of a preservative in pharmaceutical compositions is well known to the skilled worker. In this connection, reference may be made to Remington: The Science and Practice of Pharmacy, 19^{th} edition, 1995.

In further embodiments, a pharmaceutical composition of the invention comprises an isotonicity agent (i.e., a pharmaceutically acceptable agent which is included in the composition for the purpose of rendering the composition isotonic). In some embodiments, the composition is administered to a subject by injection. Relevant isotonicity agents include agents selected from the group consisting of: salts (e.g., sodium chloride), sugars and sugar alcohols, amino acids (including glycine, arginine, lysine, isoleucine, aspartic acid, tryptophan and threonine), alditols (including glycerol, propyleneglycol (i.e. 1,2-propanediol), 1,3-propanediol and 1,3-butanediol), polyethylene glycols (including PEG400) and mixtures thereof. Suitable sugars include mono-, di- and polysaccharides, and water-soluble glucans, such as fructose, glucose, mannose, sorbose, xylose, maltose, lactose, sucrose, trehalose, dextran, pullulan, dextrin, cyclodextrin, soluble starch, hydroxyethyl starch and carboxymethylcellulose sodium salt. In some embodiments sucrose may be employed. Suitable sugar alcohols include hydroxylated C₄-C₈ hydrocarbons, including mannitol, sorbitol, inositol, galacititol, dulcitol, xylitol and arabitol. In some embodiments mannitol may be employed. The sugars or sugar alcohols mentioned above may be used individually or in combination. There is no fixed limit to the amount of isotonicity agent used, as long as it is soluble in the liquid formulation, establishes isotonicity and does not adversely effect the stability of the composition. The concentration of isotonicity agent (e.g. sugar or sugar alcohol) in the final liquid composition may be, e.g., from about 1 mg/ml to about 150 mg/ml, such as from 1 mg/ml to 50 mg/ml. In particular embodiments, the concentration may be from 1 mg/ml to 7 mg/ml, or from 8 mg/ml to 24 mg/ml, or from 25 mg/ml to 50 mg/ml. The use of an isotonicity agent in pharmaceutical compositions is well known to the skilled person. In this connection, reference may be made to Remington: The Science and Practice of Pharmacy, 19^{th} edition, 1995.

In further embodiments of pharmaceutical compositions of the invention, the composition comprises a chelating agent. Relevant chelating agents include salts of ethylenediaminetetraacetic acid (EDTA), citric acid or aspartic acid, and mixtures thereof. The chelating agent may suitably be present in the final liquid composition in a concentration of from 0.1 mg/ml to 5 mg/ml, such as from 0.1 mg/ml to 2 mg/ml, or from 2 mg/ml to 5 mg/ml. The use of a chelating agent in pharmaceutical compositions is well-known to the skilled worker. In this connection, reference may be made to Remington: The Science and Practice of Pharmacy, 19^{th} edition, 1995.

In further embodiments of pharmaceutical compositions of the invention, the composition comprises a stabilizer. The use of a stabilizer in pharmaceutical compositions is well-known to the skilled worker, and in this connection reference may be made to Remington: The Science and Practice of Pharmacy, 19th edition, 1995. Particularly useful pharmaceutical compositions of the invention are stabilized liquid compositions with therapeutically active components that include a compound of the invention (e.g., a peptide of the invention) that may otherwise possibly exhibit aggregate formation during storage in a liquid medium. In this context, “aggregate formation” refers to physical interactions between the peptide molecules that result in formation of larger assemblies that undergo some degree of visible precipitation from the solution. As used herein, “during storage in a liquid medium” refers to the storage of a liquid composition that, once prepared, is not necessarily immediately administered to a subject. Instead, following preparation, it may be packaged for storage, either in a liquid form, in a frozen state, or in a dried form for later reconstitution into a liquid form or other form suitable for administration to a subject. As used herein, “dried form” refers to an initially liquid pharmaceutical composition or formulation that has been dried by freeze-drying (i.e., lyophilization), by spray-drying or by air-drying. Aggregate formation by a peptide during storage of a liquid pharmaceutical composition thereof can adversely affect biological activity of the peptide in question, resulting in a loss of therapeutic efficacy of the pharmaceutical composition. Furthermore, aggregate formation may cause other problems, such as blockage of tubing, membranes, or pumps if such a peptide-containing pharmaceutical composition is administered using an infusion system. Thus, peptides of the invention may be beneficial in overcoming these problems.

Examples of stabilizers appropriate for incorporation in pharmaceutical compositions of the invention include, but are not limited to, the following: amino acids in their free base form or salt form, e.g. amino acids carrying a charged side chain, such as arginine, lysine, aspartic acid or glutamic acid, or amino acids such as glycine or methionine (in that incorporation of methionine may additionally inhibit oxidation of methionine residues in peptides comprising at least one methionine residue susceptible to such oxidation); certain polymers (e.g., polyethylene glycols (such as PEG 3350), polyvinylalcohol (PVA), polyvinylpyrrolidone (PVP), and carboxy-/hydroxycellulose and derivatives thereof); cyclodextrins; sulfur-containing substances (such as monothioglycerol, thioglycolic acid and 2-methylthioethanol); and surfactants (such as non-ionic surfactants, including non-ionic surfactants of the Poloxamer or Polysorbate (Tween) types. The use of a surfactant in pharmaceutical compositions is well known to the skilled worker. In this connection, reference may be made to Remington: The Science and Practice of Pharmacy, 19^{th} edition, 1995.

Additional types of constituents may also be present in pharmaceutical compositions of the present invention. Non-limiting examples of classes of such constituents include wetting agents, emulsifiers, antioxidants, bulking agents, oleaginous vehicles and proteins (e.g., human serum albumin or gelatin).

Pharmaceutical compositions of the invention may be administered to a patient in need of such treatment at various sites, for example administration at sites which bypass absorption, such as in an artery or vein or in the heart, and at sites which involve absorption, such as in the skin, under the skin, in a muscle or in the abdomen. More generally, administration of pharmaceutical compositions according to the invention may be by a variety of routes of administration, such as or example parenteral, epidermal, dermal or transdermal routes. In some embodiments, other routes such as lingual, sublingual, buccal, oral, vaginal or rectal may be useful.

Compositions of the invention may be administered in various dosage forms, for example solutions, suspensions or emulsions, and are useful in the formulation of controlled-, sustained-, protracted-, retarded- or slow-release drug delivery systems. More specifically, but not exclusively, pharmaceutical compositions of the invention are useful in connection with parenteral controlled-release and sustained-release systems, well known to those skilled in the art. General reference may be made in this connection to Handbook of Pharmaceutical Controlled Release (Wise, D. L., ed., Marcel Dekker, New York, 2000) and Drugs and the Pharmaceutical Sciences vol. 99: Protein Formulation and Delivery (MacNally, E. J., ed., Marcel Dekker, New York, 2000).

Parenteral administration (of a liquid pharmaceutical composition of the invention) may be performed, for example, by subcutaneous, intramuscular, intraperitoneal or intravenous injection by means of a syringe, suitably a pen-like syringe. Alternatively, parenteral administration can take place by means of an infusion pump, e.g. in the form of a device or system borne by a subject or patient and comprising a reservoir containing a liquid composition of the invention and an infusion pump for delivery/administration of the composition to the subject or patient, or in the form of a corresponding miniaturized device suitable for implantation within the body of the subject or patient.

The term “stabilized composition” as employed herein refers to a composition having increased physical stability, increased chemical stability or increased physical and chemical stability. The term “physical stability” as used herein refers to a measure of the tendency of a peptide (e.g., a compound of the invention) to form soluble or insoluble aggregates of the peptide, for example as a result of exposure of the peptide to stresses and/or interaction with interfaces and surfaces that are destabilizing, such as hydrophobic surfaces and interfaces. Physical stability of aqueous peptide compositions may be evaluated by means of visual inspection and/or turbidity measurements after exposing the composition, filled in suitable containers (e.g. cartridges or vials), to mechanical/physical stress (e.g. agitation) at different temperatures for various time periods. A composition may be classified as physically unstable with respect to peptide aggregation when it exhibits visual turbidity. Alternatively, the turbidity of a composition can be evaluated by simple turbidity measurements well-known to the skilled person. Physical stability of an aqueous peptide composition can also be evaluated by using an agent that functions as a spectroscopic probe of the conformational status of the peptide. The probe is preferably a small molecule that preferentially binds to a non-native conformer of the peptide. One example of such a small-molecular spectroscopic probe is Thioflavin T, which is a fluorescent dye that has been widely used for the detection of amyloid fibrils. In the presence of fibrils, and perhaps also other peptide configurations, Thioflavin T gives rise to a new excitation maximum at about 450 nm and enhanced emission at about 482 nm when bound to a fibril form of a peptide. Unbound Thioflavin T is essentially non-fluorescent at the wavelengths in question.

The term “chemical stability” as used herein refers to stability of a peptide with respect to covalent chemical changes in the peptide structure that lead to formation of chemical degradation products with potentially lower biological potency and/or potentially increased immunogenicity compared to the native peptide structure. Various chemical degradation products can be formed, depending on the type and detailed nature of the native peptide and the environment to which the peptide is exposed. In practise, elimination of chemical degradation in peptide compositions in general cannot be avoided completely, and the formation of increasing amounts of chemical degradation products is often seen during storage and use of such compositions, as is well-known to the person skilled in the art. Many peptides are susceptible to a degradation process in which the side-chain amide group in glutaminyl or asparaginyl residues is hydrolysed to form a free carboxylic acid. Other degradation pathways involve formation of high-molecular-weight transformation products in which two or more peptide molecules become covalently bound to each other through transamidation and/or disulfide interactions, leading to formation of covalently bound oligomer and polymer degradation products (see, e.g., Stability of Protein Pharmaceuticals, Ahern. T. J. and Manning M. C., Plenum Press, New York 1992). Oxidation (e.g., of methionine residues) is another form of chemical degradation of peptides. The chemical stability of a peptide composition may be evaluated by measuring the amounts of chemical degradation products at various time-points after exposure to different environmental conditions (for example, formation of degradation products may often be accelerated by increasing temperature). The amount of each individual degradation product may be determined by separation of the degradation products on the basis of molecular size and/or charge using various chromatographic techniques (e.g. SEC-HPLC and/or RP-HPLC).

The chemical instability of glucagon per se at low pH is mainly due to isomerisation and cleavage of aspartic acid residues, deamidation of glutamine residues and oxidation of methionine. Generally speaking, Asn and Gin deamidation occurs at high pH, with significant rates at physiological pH around pH 7.4 via a cyclic imide ring intermediate which can open to create L-Asp and L-isoAsp or L-Glu and L-isoGlu, respectively. The cyclic imide ring intermediate also may lead to the formation of small amounts of the corresponding D-isomers, indicating a slow racemisation of the cyclic imide. At pH values below physiological pH, the rate of deamidation of Asn and Gin is reduced, but the rate of formation of a cyclic imide from Asp and Glu, and hence isomerisation, increases with decreasing pH. Cyclic imide formation is greatest between pH 4 and pH 6. Formation of the cyclic imide intermediate can also result in cleavage of the peptide sequence.

As outlined above, a “stabilized composition” may thus refer to a composition with increased physical stability, or increased chemical stability, or increased physical and chemical stability. In general, a composition should be stable during use and storage (in compliance with recommended use and storage conditions) at least until the specified expiration date is reached.

In certain embodiments of pharmaceutical compositions of the invention (e.g., liquid compositions) the composition is stable for at least 2 weeks of usage and for at least 6 months of storage. In further embodiments, the composition is stable for at least 2 weeks of usage and for at least one year of storage. In still further embodiments, the composition is stable for at least 2 weeks of usage and for at least two years of storage. In other embodiments, the composition is stable for at least 4 weeks of usage and for at least two years of storage, or even for at least 4 weeks of usage and for more than 3 years of storage. Particularly useful embodiments of such pharmaceutical compositions of the invention are stable for at least 6 weeks of usage and for at least 3 years of storage. In this regard, the term “usage” for the purposes of this paragraph refers to taking the pharmaceutical composition out of storage for the purpose of employing the composition for therapeutic purposes, and thereby subjecting it to varying ambient conditions (conditions of light, dark, temperature, agitation etc.), whilst the term “storage” for the purposes of this paragraph refers to storage under non-agitated conditions in a refrigerator or freezer at a temperature not exceeding about 5° C. The skilled worker will understand the typical range of usage and storage conditions that these pharmaceutical compositions may be subjected to.

### EXAMPLES

The following examples are provided to illustrate preferred aspects of the invention and are not intended to limit the scope of the invention. The glucagon analogues administered according to the dosage regimes described herein can be made according to the methods such as solid phase peptide synthesis described in WO 2014/016300, the content of which is expressly incorporated by reference in its entirety.

Methods

Data:

Insulin and glucagon PK models were used in combination with a validated glucose-insulin-glucagon PD model to simulate data from seven virtual type 1 diabetes patients (12). MATLAB 2016b (The MathWorks, Inc., Natick, Mass.) was used for model implementation and simulations.

The population of virtual patients described seven “real” adults (4 females, age range: 19-64 years, BMI range: 20.0-25.4 kg/m²) with insulin pump-treated type 1 diabetes, who previously had participated in a study investigating the glucose response to different mini-doses of glucagon during insulin-induced mild hypoglycaemia (13). Patients were in good glycaemic control (HbA1c range: 6.1-7.4%) and had no endogenous insulin production (13).

The PD model is an extension of Hovorka's glucoregulatory model with the effects of glucagon on the endogenous glucose production (Hovorka et al., 2004) and was validated in a previous study (Wendt et al. 2017). The PK models assumed that changes in insulin and glucagon concentrations were only due to the administered drugs: insulin aspart (NovoRapid®, Novo Nordisk) and glucagon (GlucaGen®, Novo Nordisk).

### Example 1: In Silico Experiments

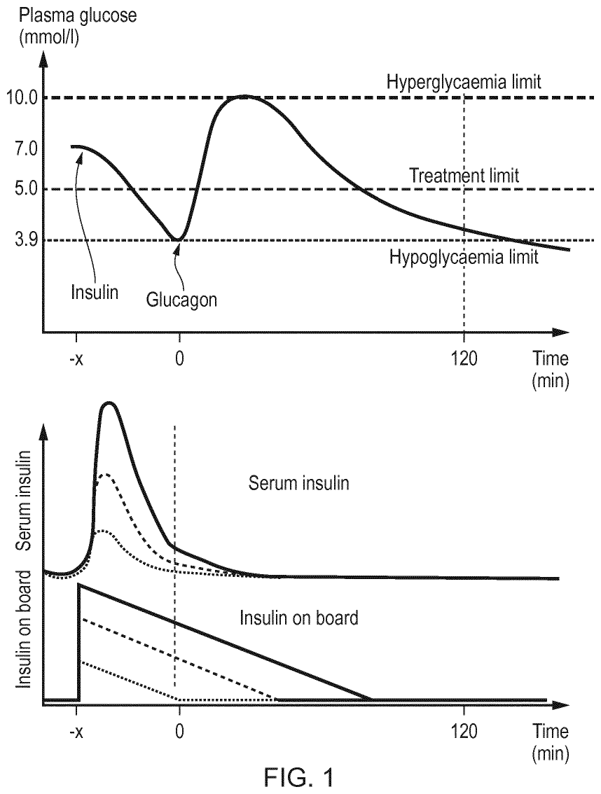

Three simulations were carried out to investigate the glucose response to different glucagon doses depending on the ambient insulin levels during insulin-induced mild hypoglycaemia (FIG. 1). In each simulation, a SC insulin bolus was administered at PG of 7.0 mmol/l, followed by a SC glucagon bolus that was administered when PG was 3.9 mmol/l. The simulation of one experiment lasted for ten hours following the insulin bolus.

The individual insulin bolus size was chosen to achieve a predefined insulin level at the time of glucagon administration. Thus, patients received different insulin boluses to achieve the same predefined insulin levels, due to differences in insulin PK/PD profiles.

For each simulation, one of the following predefined insulin levels was achieved when PG was 3.9 mmol/l:

1) Ratio of actual to baseline serum insulin concentration (se/ba-insulin): 1.0, 1.25, 1.5, 1.75, 2.0, 2.25, 2.5, 3.0, 3.5 or 4.0.

2a) Insulin on board (IOB): 0.0, 0.25, 0.5, 0.75, 1, 1.5, 2, 2.5, 3, or 3.5 U. 2b) Percentage of insulin on board to total daily insulin dose (IOB/TDD): 0, 1, 2, 3, 4, 5, 6, 7, 8, or 10%.

The insulin PK model was used to estimate the actual serum insulin level which was divided by the individual baseline level before the insulin bolus was given (se/ba-insulin). A linear function of patient's insulin action time was used to estimate IOB. TDD was an average of seven days.

In all experiments, when PG reached 3.9 mmol/l, one of following 17 glucagon boluses was administered SC: 25, 50, 75, 100, 125, 150, 175, 200, 250, 300, 400, 500, 1000, 1500, 2000, or 2500 μg.

Treatment assessment: For each experiment, the success of a glucagon dose in treating mild hypoglycaemia but avoiding rebound hyperglycaemia was evaluated based on three criteria: peak PG≥5.0 mmol/l (PG≥5), peak PG≤10.0 mmol/l (PG≤10) and PG≥3.9 mmol/l for 120 min after the glucagon bolus (PG₁₂₀≥3.9) (FIG. 1). The success rate of a glucagon bolus in achieving each of these criteria was calculated at various insulin levels. For each combination of glucagon dose and insulin level, the overall treatment success was calculated as a weighted harmonic mean (H) of the three criteria:

$$H = \frac{1}{\frac{0.4}{{S}_{PG \geq 5}} + \frac{0.4}{{S}_{PG \leq 10}} + \frac{0.2}{{S}_{{PG}_{120} \geq 3.9}}} ,$$

where S is the success rate, equal to the number of subjects fulfilling a criterion divided by the total number of subjects. Arbitrarily, the weighted harmonic mean prioritises the criteria for peak PG (PG≥5 and PG≤10) higher than the PG level two hours after dose (PG₁₂₀≥3.9), since we consider the acute rescue of hypoglycaemia and the avoidance of rebound hyperglycaemia to be more important than the duration of the anti-hypoglycaemic effect. For each insulin level, the lowest glucagon dose with the highest H-value was the optimum bolus.

Results

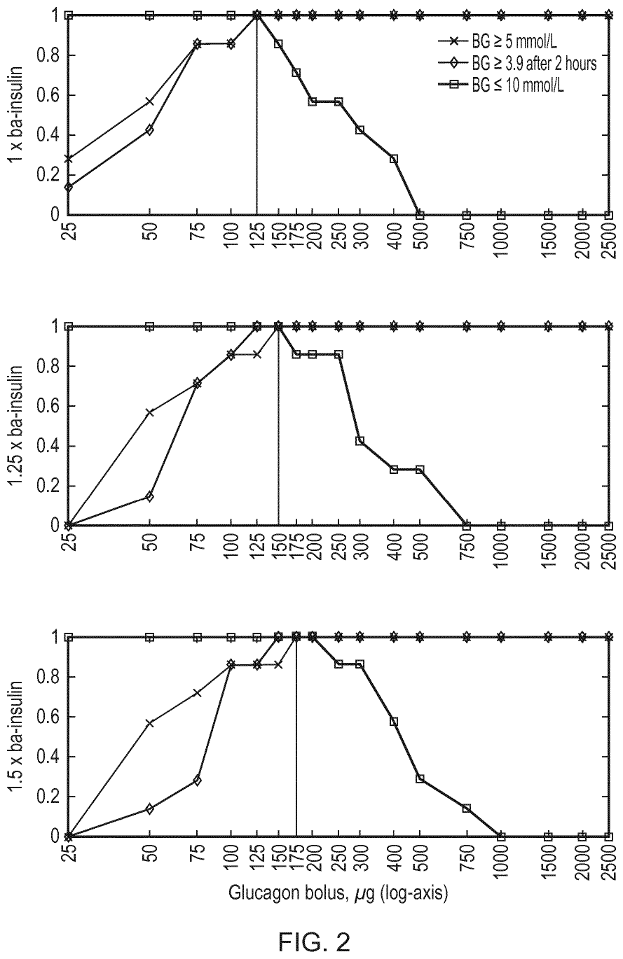

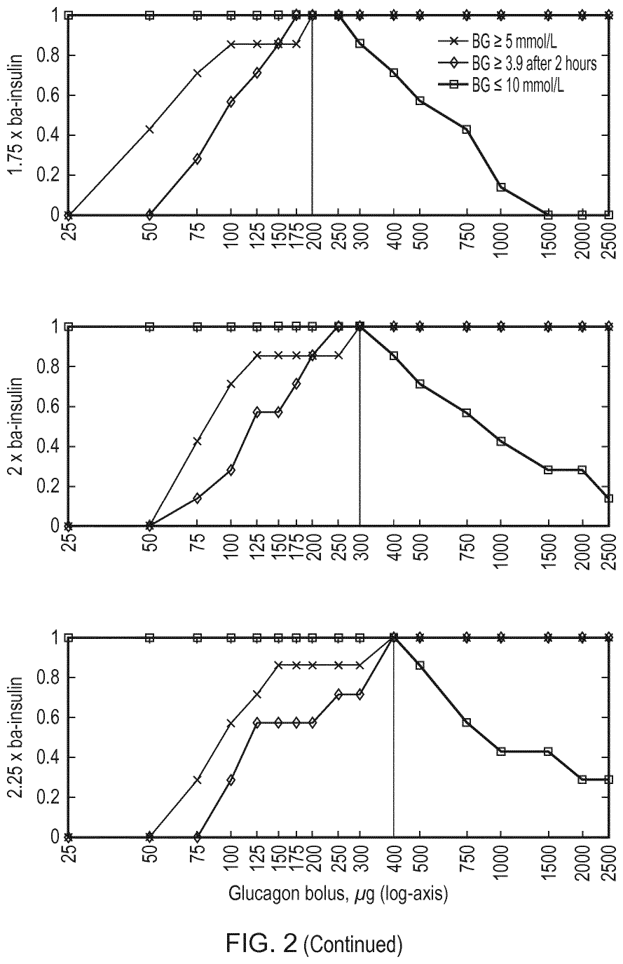

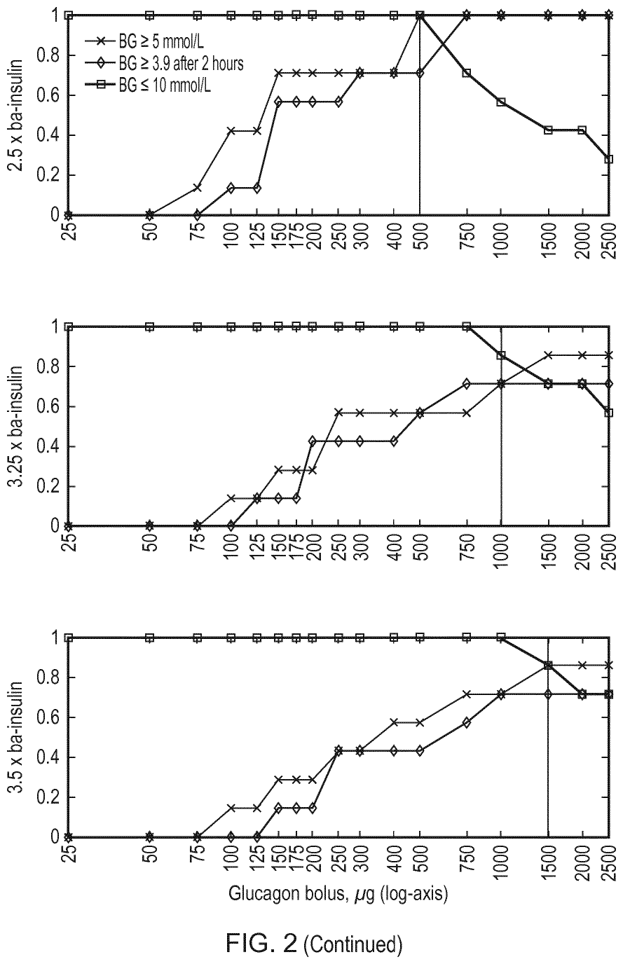

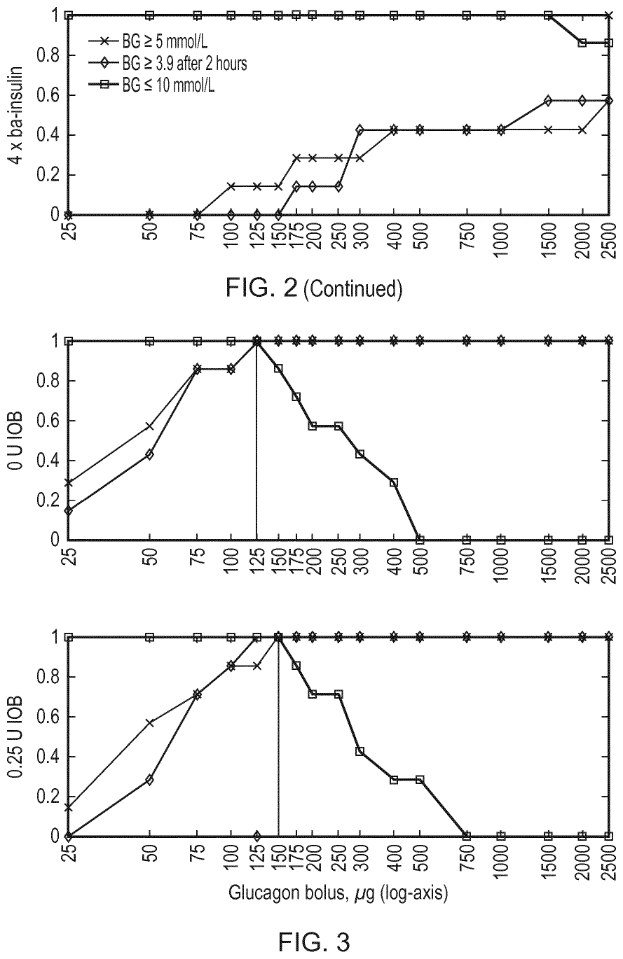

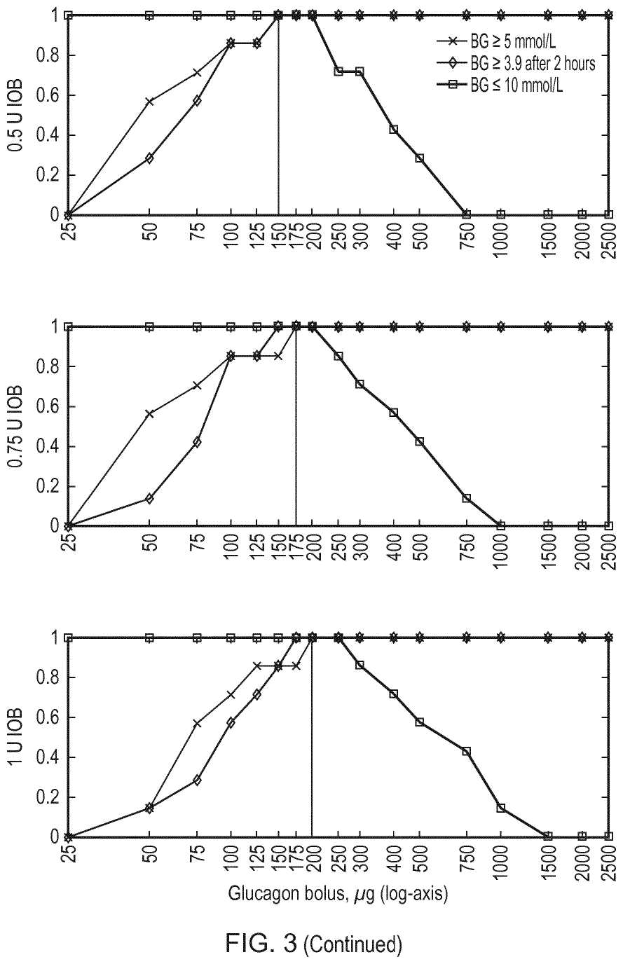

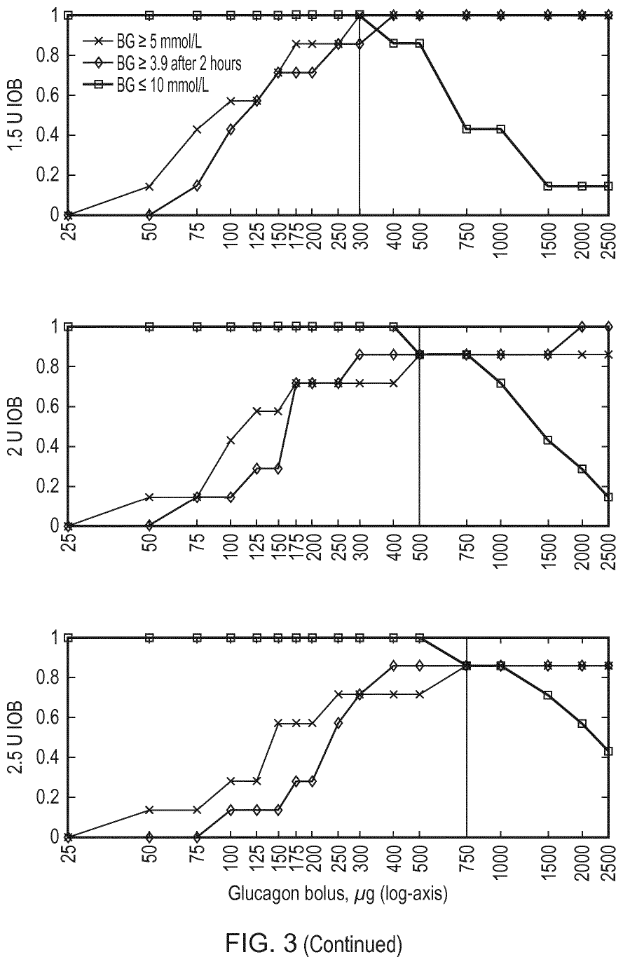

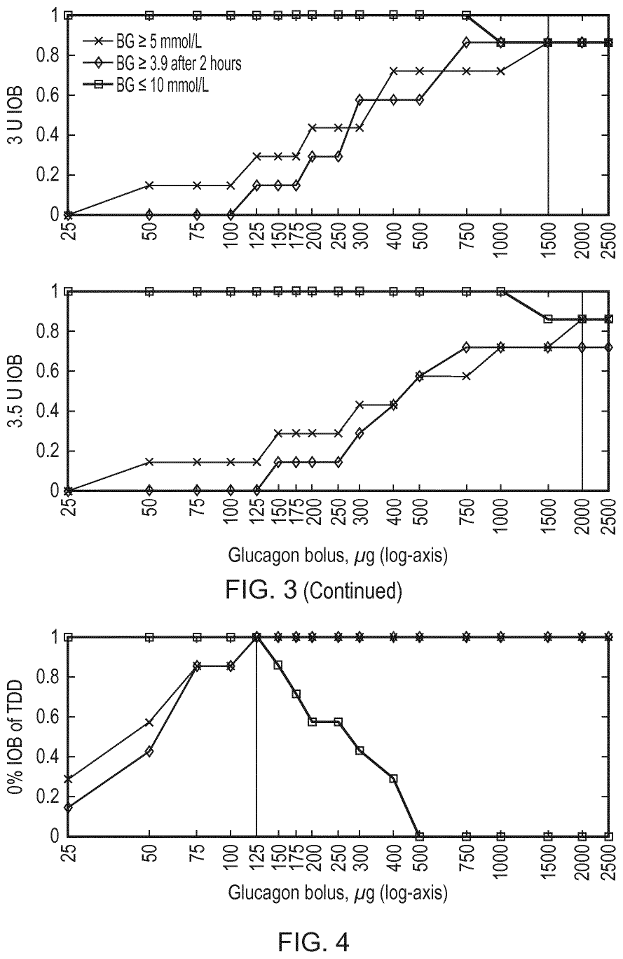

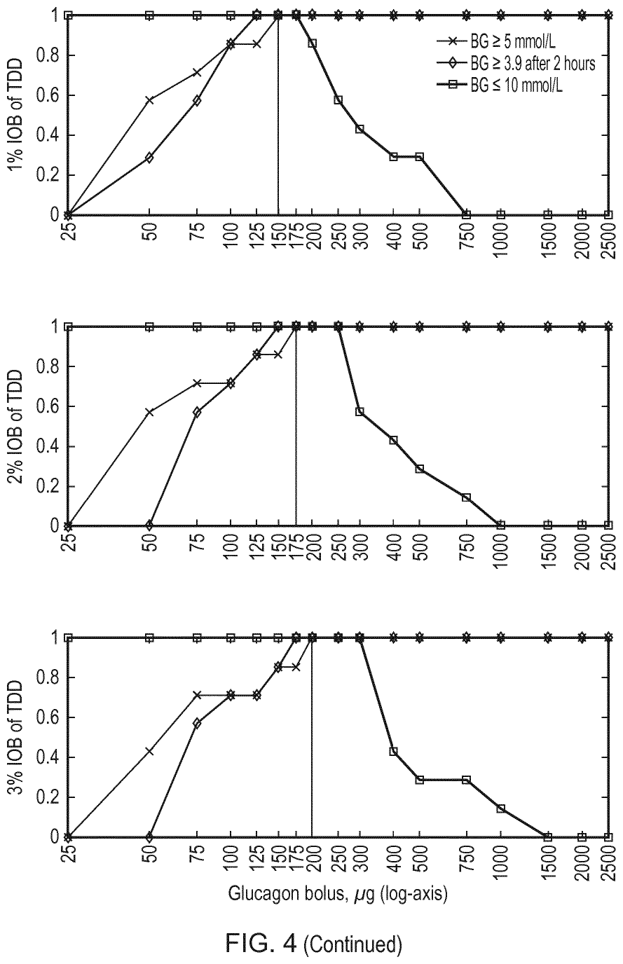

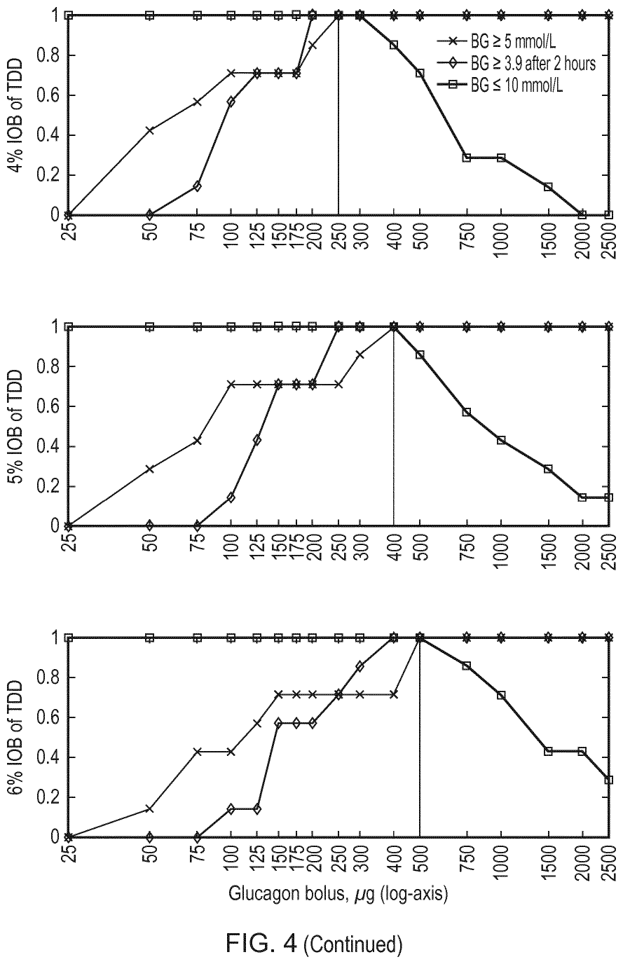

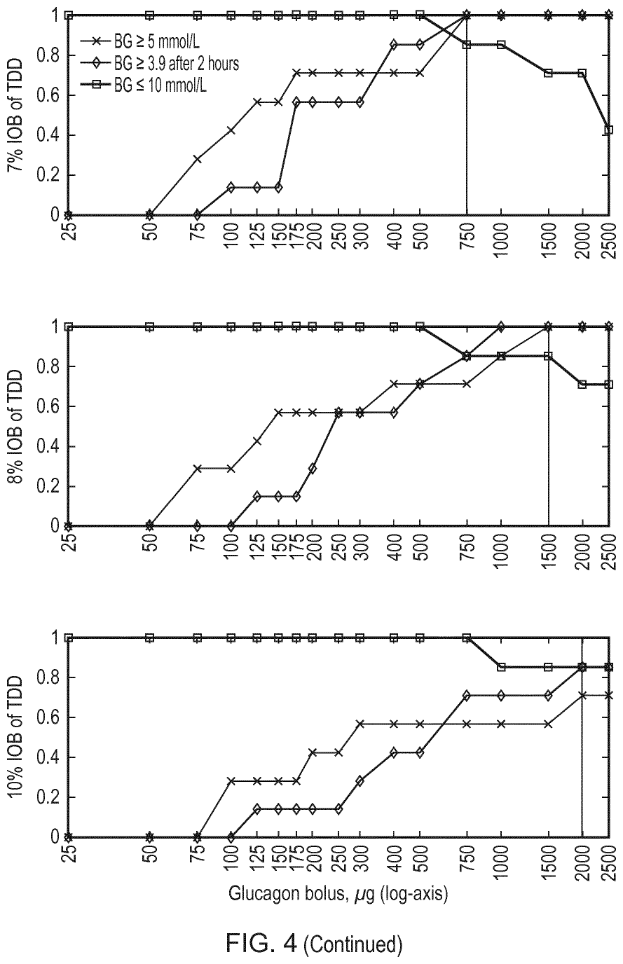

FIGS. 2-4 show the proportion of patients achieving the predefined treatment criteria as functions of the glucagon dose, stratified by se/ba-insulin (FIG. 2), IOB (FIG. 3), and IOB/TDD (FIG. 4). The proportion of patients achieving the criterion of PG≥5 (green line) and of PG₁₂₀-3.9 (red line) increased with increasing glucagon doses. The curves for the PG≥5 criterion were left-shifted compared to the curves for the PG₁₂₀≥3.9 criterion, meaning that less glucagon was needed to fulfil the criterion of PG≥5 compared to PG₁₂₀≥3.9. On the other hand, the proportion of patients avoiding rebound hyperglycaemia, PG≤10, declined with increasing glucagon doses (blue line). For instance, when patients had a PG of 3.9 mmol/l and IOB of 1.5 U, a glucagon dose of 100 μg would increase PG≥5.0 mmol/l in less than 60% of patients, keep PG≥3.9 mmol/l for two hours in more than 40% of patients, and keep PG≤10 mmol/l in all patients.

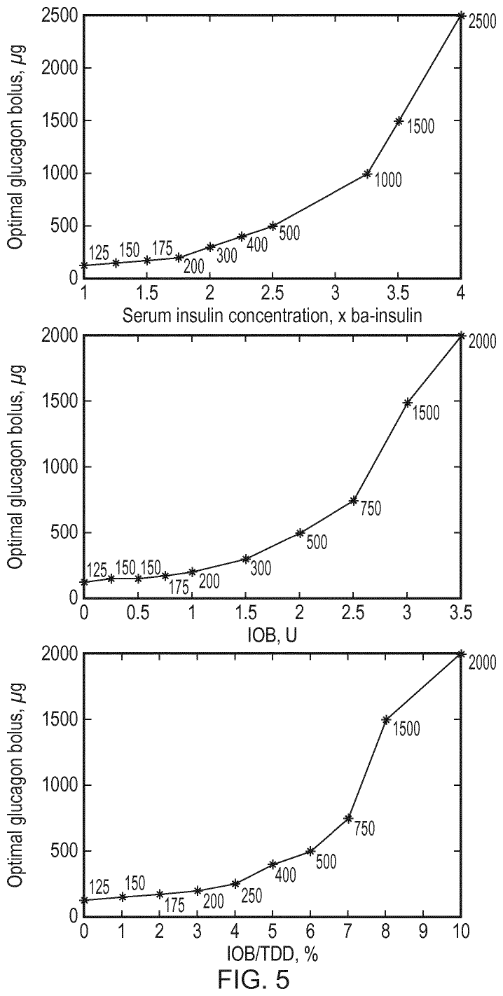

FIG. 5 shows the optimum glucagon dosing regimens for treatment of mild hypoglycaemia in the virtual population as a function of insulin levels extracted from FIGS. 2-4 (vertical black lines). The relationship between insulin level and the corresponding optimum glucagon dose could be approximated by an exponential function, regardless of the method used for estimating insulin levels. A 125 μg glucagon dose was needed to optimally treat mild hypoglycaemia when insulin levels were equal to baseline levels. In contrast, glucagon doses >500 μg were needed when serum insulin exceeded 2.5 times baseline insulin concentrations, IOB were above 2.0 U or IOB/TDD were above 6%.

Discussion

In this in silico study, we used a validated PK/PD model to develop optimum glucagon dosing regimens to treat mild hypoglycaemia at varying levels of serum insulin ratio (i.e. the actual serum insulin level divided by patients' baseline insulin level before the insulin bolus), “insulin on board” and percentage of “insulin on board” to total daily insulin use in patients with type 1 diabetes. The anti-hypoglycaemic effect of glucagon was highly dependent on ambient insulin levels. El Youssef et al. previously showed this relation in vivo by quantifying the glycaemic effects of glucagon at various insulin levels (El Youssef et al., 2014). Notably, the PK/PD model used in the present study was able to replicate the findings by El Youssef et al. with simulations (Wendt et al., 2017). Furthermore, the PD model was validated using data from another cross-over in vivo study with three different SC injections of glucagon for treatment of insulin-induced mild hypoglycaemia (Ranjan et al., 2016). Therefore, the model for estimating the optimum glucagon dose for treatment of mild hypoglycaemia at varying levels of insulin is valid.

The strength of in silico studies is the ability to simulate large scale cross-over trials that are not feasible in real-life settings. In this study, we estimated the optimum glucagon dose at varying insulin levels based on virtual patients, each undergoing 170 cross-over visits per study, resulting in 510 simulations per patient. An optimum glucagon dose was defined as increasing PG from 3.9 mmol/l to a peak between 5.0 and 10.0 mmol/l, and sustaining PG above 3.9 mmol/l for at least 120 minutes following the glucagon bolus. These success criteria were arbitrarily set as no consensus exists regarding post-rescue glucose excursions or postprandial glucose excursions. We based our criteria on the recommendations of American Diabetes Association that were considered clinically reasonable in most patients. However, not all criteria could be achieved in all patients with the same glucagon dose. We considered the acute rescue of mild hypoglycaemia and the following avoidance of rebound hyperglycaemia to be more important than the duration of the anti-hypoglycaemic effect. This priority of peak PG over the 2-hour PG level was applied because patients will benefit from the acute rescue and still have time to avoid subsequent hypoglycaemia by suspending their insulin infusion and/or consuming carbohydrates. Alternatively, a second bolus of glucagon could be given which has shown to give similar glucose response as the first glucagon bolus.

The glucagon dosing regimens were stratified in relation to different methods of estimating ambient insulin levels. IOB was included because no real-time monitors of serum insulin concentrations are currently available. For decades, insulin pumps with bolus calculators have used IOB feedback as standard to prevent insulin stacking. Depending on the manufacturer, the bolus calculators estimate IOB differently, i.e. using a linear or a curvilinear time profile and most bolus calculators use a curvilinear time profile because it resembles the insulin time-action profile (Zisser et al., 2008). However, the linear approach was chosen due to the unambiguous implementation compared with the curvilinear functions. Further, we consider the differences in IOB time profiles to be negligible for the success of glucagon treatment.

In this study, an exponential relationship between the optimum glucagon doses to treat mild hypoglycaemia and the ambient insulin levels was found. However, the relationship was approximately linear in ranges of serum insulin from 1 to 2 times basal insulin levels, IOB from 0 to 2 U, and IOB/TDD from 0 to 5%. The lowest glucagon dose to optimally treat mild hypoglycaemia was 125 μg when actual insulin levels were equal to baseline levels. In contrast, at very high insulin levels (se/ba-insulin>3×, IOB≥3 U, IOB/TDD>8%), the optimum glucagon doses exceeded the amount (1000 μg) normally used for treating severe hypoglycaemia. Further, at some point the estimated optimum glucagon dose was, in our opinion, too high (>500 μg) as treatment option for mild hypoglycaemia, especially due to the increased risk of side effects. In the present study, we found that 500 μg glucagon was needed if serum insulin was 2.5 times basal insulin levels, IOB was 2 U, or IOB was 6% of TDD. Therefore, if patients have mild hypoglycaemia, but insulin levels above these critical limits, ingestion of carbohydrates rather than mini-dose glucagon may be a better treatment for restoring PG.

The same success criteria were applied for optimum glucagon dosing to the results of a previous in vivo dose finding study. Here in a comparison of glucagon doses, 200 and 300 μg had almost similar success rate to restore mild hypoglycaemia with an average IOB of 0.9 U or IOB/TDD of 2%. The lowest optimum dose was similar to the dose suggested in the current in silico (FIG. 5).

Glucagon is currently only available in 1 mg vials and has to be reconstituted immediately before use. However, based on the methods and medical uses of the present invention, stable soluble glucagon formulations may also be used for the mini dosing of glucagon to alleviate mild hypoglycaemia, as opposed to only rescue dosing to treat severe hypoglycaemia. An advanced bolus calculator advising for insulin injections, carbohydrate intake and glucagon injections could account for side effects, treatment success, and IOB; might provide a good option for prevention and treatment of hypoglycaemia and leading to improved glucose control.

This is the first study to propose a dosing regimen of glucagon in an open-loop setting using simulations. This validates low dose glucagon as an alternative to oral carbohydrate intake in treatment of mild hypoglycaemia. Accordingly, low dose glucagon treatment, optionally when used in combination with an advanced bolus calculator, may provide more predictable glucose responses than oral carbohydrate ingestion in treatment of mild hypoglycaemia, and may also reduce the risk of overeating and post-rescue hyperglycaemia.

In this study, a mathematical PK/PD model was used to develop insulin-dependent optimum glucagon dosing regimens for treatment of insulin-induced mild hypoglycaemia.

The glucagon doses depend on insulin levels evaluated as serum insulin concentration normalised to basal, insulin on board and ratio of insulin on board to TDD. The regimens were based on simulations of glucagon doses ranging from 25 to 2500 μg and insulin doses yielding predefined insulin levels when blood glucose reached the hypoglycaemia threshold.

### Example 2: Feasibility Study of the Mini-Dose Glucaon Concept in the Treatment of Hypoglycemia in Type 1 Diabetes

Rationale: Several studies have shown that the glucose response to mini-dose glucagon is influenced by ambient insulin levels. A fixed glucagon dose to treat hypoglycemia may not be sufficient when insulin levels are high and may fail to restore euglycaemia. We recently performed a simulation study showing the glucose response to glucagon at various ambient insulin levels (Ranjan et al. Relationship between Optimum Mini-doses of Glucagon and Insulin Levels when Treating Mild Hypoglycaemia in Patients with Type 1 Diabetes—A Simulation Study. In: Basic & Clinical Pharmacology & Toxicology Online, Vol. 122, No. 3, 03.2018, p. 322-330). We concluded that the glucagon dose to optimally treat mild hypoglycaemia depends exponentially on insulin levels, regardless of how insulin was estimated (either as serum insulin, insulin-on-board (IOB), and the ratio of insulin-on-board to total daily insulin (IOB/TDD)). The optimal dose was defined as a dose, which could treat mild hypoglycemia without risking subsequent hyperglycemia or hypoglycemia. We want to test whether this insulin-dependent glucagon dosing regimen developed can be applied in a clinical setting.

Aim:

To confirm that our insulin-dependent glucagon dosing regimen can provide sufficient glucose elevating effects during mild hypoglycemia regardless of the ambient insulin levels.

Design:

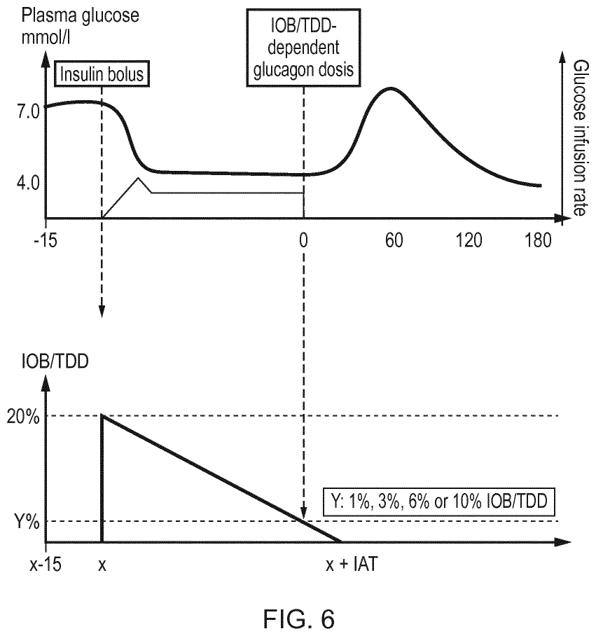

This is a randomized single-blinded controlled cross-over study. Patients will complete four study visits at the research facility in random order. On each visit, s.c. insulin bolus equal to 20% of patient's total daily insulin dose (IOB/TDD=20%) will be given in a fasting state in the morning, while a variable iv glucose infusion rate is given to maintain target PG level of 4.0 mmol/l until IOB/TDD achieves the predefined levels of 1, 3, 6 or 10%. The IOB follows a linear decay that will be zero depending on patient's insulin action time (IAT), which is close to reality. Once the predefined IOB/TDD level is reached, the glucose infusion rate is stopped and s.c. 100 μg glucagon is injected. PG levels will be monitored for another 180 min (FIG. 6).

Endpoints:

Percentages of patients that achieve the success criteria of optimal post-glucagon treatment, i.e. PG of 4-10 mmol/l for two hours after glucagon injection.

Statistical Issues:

Twenty-one patients should be included if 90% of the patients have to meet the predefined success criteria stated in the simulation study with a power of 80%.

Significance:

A fixed glucagon dosing regimen cannot guarantee optimal glucose recovery due to several factors (e.g. insulin, plasma glucose level) that affect glucagon efficacy. A variable glucagon dosing regimen depending on these factors therefore provides the possibility of improved glucose control compared with available regimens and that further studies to demonstrate the feasibility, safety and efficacy of such a regimen in real life settings are supported by these results. The present inventors believe that such an open-loop dual-hormone system may be much cheaper but still perform equally to the dual-hormone closed-loop systems.

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All documents cited herein are expressly incorporated by reference.

### REFERENCES

El Youssef et al., Quantification of the glycemic response to microdoses of subcutaneous glucagon at varying insulin levels. Diabetes Care 2014; 37:3054-60.
Zisser et al., Bolus calculator: a review of four “smart” insulin pumps. Diabetes Technol. Ther. 2008; 10:441-4.
Wendt et al., Cross-Validation of a Glucose-Insulin-Glucagon Pharmacodynamics Model for Simulation Using Data From Patients With Type 1 Diabetes. J Diabetes Sci. Technol. 2017.
Ranjan et al., Effects of subcutaneous, low-dose glucagon on insulin-induced mild hypoglycaemia in patients with insulin pump treated type 1 diabetes. Diabetes Obes. Metab. 2016; 18(4):410-8.
Hovorka et al., Nonlinear model predictive control of glucose concentration in subjects with type 1 diabetes. Physiol. Meas. England; 2004; 25:905-20.
Wendt et al., Simulating clinical studies of the glucoregulatory system: in vivo meets in silico. Technical University of Denmark, Tech. Rep. 1, February 2017.
Ranjan et al. Relationship between Optimum Mini-doses of Glucagon and Insulin Levels when Treating Mild Hypoglycaemia in Patients with Type 1 Diabetes—A Simulation Study. In: Basic & Clinical Pharmacology & Toxicology Online, Vol. 122, No. 3, 03.2018, p. 322-330.

1. An automated or computer implemented method for determining a dose for a glucagon bolus for administration to a patient with diabetes for treating mild or moderate hypoglycaemia, the method comprising:
(a) determining an ambient insulin level for the patient, wherein the ambient insulin level is directly measured by a blood sample, measured by an insulin sensor and/or approximated by active insulin on board;
(bi) using the ambient insulin level to determine the dose for a glucagon bolus to treat mild or moderate hypoglycaemia while reducing the risk of, or avoiding, rebound hyperglycaemia according to treatment criteria (1) to increase plasma glucose (PG)≥5 mmol/l, (2) to have a peak plasma glucose (PG)≤10 mmol/l, and (3) to keep plasma glucose (PG)≥3.9 mmol/l for 120 min after the glucagon bolus;
(bii) using pharmacokinetic/pharmacodynamics (PK/PD) models for simulation of a virtual patient population of diabetes patients receiving the glucagon bolus to correct insulin-induced mild or moderate hypoglycaemia, wherein the dose of the glucagon bolus is the lowest glucagon dose yielding the maximal weighted success rate in the population calculated according to said treatment criteria; and
(c) selecting the lowest dose for the glucagon bolus that provides the maximal weighted success rate according to the treatment criteria; and
(d) optionally administering the glucagon bolus to the patient to treat the hypoglycaemia.

2. A glucagon for use in a method of treating mild or moderate hypoglycaemia in a patient with diabetes, wherein the method comprises calculating a dose for a glucagon bolus using an automated or computer implemented method which comprises:
(a) determining an ambient insulin level for the patient, wherein the ambient insulin level is directly measured by a blood sample, measured by an insulin sensor and/or approximated by active insulin on board;
(bi) using the ambient insulin level to determine the dose for a glucagon bolus to treat mild or moderate hypoglycaemia while reducing the risk of, or avoiding, rebound hyperglycaemia according to treatment criteria (1) to increase plasma glucose (PG)≥5 mmol/l, (2) to have a peak plasma glucose (PG)≤10 mmol/l, and (3) to keep plasma glucose (PG)≥3.9 mmol/l for 120 min after the glucagon bolus;
(bii) using pharmacokinetic/pharmacodynamics (PK/PD) models for simulation of a virtual patient population of diabetes patients receiving the glucagon bolus to correct insulin-induced mild or moderate hypoglycaemia, wherein the dose of the glucagon bolus is the lowest glucagon dose yielding the maximal weighted success rate in the population calculated according to said treatment criteria; and
(c) selecting the lowest dose for the glucagon bolus that provides the maximal weighted success rate according to the treatment criteria; and
(d) administering the glucagon bolus to the patient to treat the hypoglycaemia.

3. The method or compound for use according to claim 1 or claim 2, wherein the patient treated for mild or moderate hypoglycaemia has type 1 diabetes.

4. The method or compound for use according to any one of claims 1 to 3, wherein the ambient insulin level is determined as a function of one or more of insulin-on-board (IOB), serum insulin level, the ratio of actual to baseline serum insulin concentration and/or percentage insulin on board to total daily insulin dosage (IOB/TDD %).

5. The method or compound for use according to any one of the preceding claims, wherein the ambient insulin level is determined as a function of the insulin-on-board (IOB) for the patient.

6. The method or compound for use according to any one of the preceding claims, wherein the method comprises determining insulin-on-board (IOB) using a bolus calculator.

7. The method or compound for use according to any one of the preceding claims, wherein the bolus calculator uses a linear or curvilinear time profile.

8. The method or compound for use according to any one of the preceding claims, wherein the method is carried out using an app on a mobile device such as a smart phone or using a device with a built in processors such as an insulin pump.

9. The method or compound for use according to any one of the preceding claims, wherein the hypoglycaemia is insulin-induced hypoglycaemia or hypoglycaemia induced by exercise, stress or illness.

10. The method or compound for use according to any one of the preceding claims, wherein the method comprises an initial step of administering insulin to the patient, optionally following the consumption of food by the patient.

11. The method or compound for use according to any one of the preceding claims, wherein the glucagon bolus is administered in an open loop setting, and optionally wherein the patient is additionally treated using insulin in an open loop setting, a closed-loop setting or in a hybrid open-loop setting.

12. The method or compound for use according to any one of the preceding claims, wherein the glucagon is human native glucagon or a glucagon analogue.

13. The method or compound for use according to claim 12, wherein the glucagon is human glucagon having the Hy-HSQGTFTSDYSKYLDSRRAQDFVQWLMNT-OH, or pharmaceutically acceptable salts and/or solvates thereof.

14. The method or compound for use according to claim 12, wherein the glucagon is a glucagon analogue is represented by the formula:
R¹—Z—R² (I)
or a pharmaceutically acceptable salt or solvate thereof;
wherein
R¹ is hydrogen-, C₁₋₄ alkyl, acetyl, formyl, benzoyl or trifluoroacetyl;
R² is —OH or —NH₂; and
Z is an amino acid sequence deriving from the sequence of formula Ia:
His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Glu-Asn-Thr (Ia)
and further comprising at least four amino acid substitutions or deletions that are only at sequence positions (designated by an X) selected from 2, 3, 4, 9, 10, 15, 16, 17, 20, 21, 24, 28 and 29, as follows:
X2 is selected from Aib and Ala;
X3 is selected from His, Pro, Dab(Ac), Dap(Ac) and Gln(Me);
X4 is DAla;
X9 is Glu;
X10 is selected from Val, Leu N-Me-Tyr and N-Me-DTyr;
X15 is Glu;
X16 is selected from Aib, Lys, Glu, Leu, Val, DVal, Phe, His, Arg, Pro, DPro, N-Me-Ser and N-Me-DSer;
X17 is selected from Ala and Ser;
X20 is selected from Glu and Lys;
X21 is selected from Glu, Lys and Ser;
X24 is selected from Lys, Ser, Glu and Ala;
X25 is selected from Arg, Lys, His, lie, Leu, Ala, Met, Cys, Asn, Val, Ser, Glu, Asp, Gin, Thr and (p)Tyr;
X28 is selected from Ser, Lys, and Glu, or is absent;
X29 is selected from Ser and Ala, or is absent.

15. The method or compound for use according to claim 12, wherein the glucagon is a glucagon analogue is HSQGTFTSDYSKYLD-Aib-ARAEEFVKWLEST (SEQ ID NO: 22) or HSQGTFTSDYSKYLD-Aib-ARAESFVKWLEST (SEQ ID NO: 16) or pharmaceutically acceptable salts and/or solvates thereof.

16. The method or compound for use according to any one of the preceding claims, wherein the glucagon dose is for subcutaneous injection or intramuscular injection.

17. The method or compound for use according to any one of the preceding claims, wherein the optimum glucagon dose is between 125 μg and 500 μg.

18. The method or compound for use according to any one of the preceding claims, wherein calculating the maximal weighted success rate uses a weighted harmonic mean (H) according to the formula:
$$H = \frac{1}{\frac{0.4}{{S}_{PG \geq 5}} + \frac{0.4}{{S}_{PG \leq 10}} + \frac{0.2}{{S}_{{PG}_{120} \geq 3.9}}}$$
and selecting the optimum dose comprises selecting the lowest glucagon dose with the highest H-value.
